(19) United States
(12) Patent Application Publication
(10) Pub. No.: US 2013/0236397 A1
(43) Pub. Date: Sep. 12, 2013

(54) HAIR SETTING COMPOSITIONS BASED ON T-BUTYL ACRYLATE AND HYDROXYALKYL METHACRYLATE

(71) Applicants: Gabi Winter, Wachenheim (DE); Matthias Laubender, Schifferstadt (DE)

(72) Inventors: Gabi Winter, Wachenheim (DE); Matthias Laubender, Schifferstadt (DE)

(73) Assignee: BASF SE, Ludwigshafen (DE)

(21) Appl. No.: 13/865,248

(22) Filed: Apr. 18, 2013

Related U.S. Application Data
(63) Continuation of application No. 12/088,716, filed on Mar. 31, 2008, now abandoned.

(30) Foreign Application Priority Data
Oct. 7, 2005 (EP) 05 109363.1

(51) Int. Cl. A61K 8/81; A61Q 5/12; A61Q 1/00; A61Q 5/06; A61K 8/04

(52) U.S. Cl. CPC A61K 8/8152; A61Q 5/06; A61K 8/046; A61Q 1/00; A61Q 5/12
USPC 424/47; 526/318.42; 424/70.16; 514/772.6

(57) ABSTRACT

The present invention relates to copolymers, which comprises, in copolymerized form, between 25 and 80% by weight of tert-butyl(meth)acrylate, 2 to 60% by weight of hydroxyalkyl(meth)acrylate, 10 to 40% by weight of an anionic or anionogenic, free-radically polymerizable, olefinically unsaturated compound which is or comprises methacrylic acid, and, if appropriate, up to 30% by weight of a further free-radically polymerizable, olefinically unsaturated compound. In addition, the present invention relates to the use of such copolymers in cosmetic preparations, and to such cosmetic preparations per se.

The present invention relates to copolymers which comprises, in copolymerized form, between 25 and 80% by weight of tert-butyl(meth)acrylate, 2 to 60% by weight of hydroxyalkyl(meth)acrylate, 10 to 40% by weight of an anionic or anionogenic, free-radically polymerizable, olefinically unsaturated compound which is or comprises methacrylic acid, and, if appropriate, up to 30% by weight of a further free-radically polymerizable, olefinically unsaturated compound. In addition, the present invention relates to the use of such copolymers in cosmetic preparations, and also to such cosmetic preparations per se.

### PRIOR ART

Stricter environmental regulations and a growing ecological awareness increasingly demand ever lower fractions of volatile organic components (VOCs) in cosmetic aerosol preparations such as, for example, aerosol hairsprays.

The VOC content in hairsprays is essentially determined by the nonaqueous solvents and the propellants. For this reason, instead of nonaqueous solvents, recourse is currently and increasingly being made to water as solvent. However, this replacement of the organic solvents has a number of problems.

Thus, formulations of the film-forming polymers known from the prior art which satisfy the corresponding VOC regulations are not, for example, sprayable, or are only sprayable following further dilution, and are thus only of limited suitability for use in hairsprays. Polymer films which are formed from such preparations sometimes do not have the required mechanical quality and thus have inadequate setting action and poor hold for the hair.

DE 1928368 describes binders for cosmetic preparations which are notable for the fact that it comprises a hydrophilic polyacrylate and/or -methacrylate. The polymeric binder is preferably a hydroxyalkyl or hydroxyalkoxyalkyl acrylate, or methacrylate with lower alkyl radicals and, if appropriate, lower alkoxy radicals, acryl- or methacrylamide, diacetoneacrylamide, methylolacrylamide or methylolmethacrylamide. Copolymers, which comprise copolymerized tert-butyl(meth)acrylates are not described.

EP-A 379 082 describes hair-setting compositions, which comprise, as film formers, copolymers based on tert-butyl acrylate and/or tert-butyl methacrylate with a K-value of from 10 to 50, which are obtainable by free-radical polymerization of from 75 to 99% by weight of tert-butyl acrylate and/or tert-butyl methacrylate as monomer A, 1 to 25% by weight of acrylic acid and/or methacrylic acid as monomer B and 0 to 10% by weight of a further free-radically copolymerizable monomer C, where the carboxyl groups of the copolymers are partially or completely neutralized by amines. Copolymers which comprise copolymerized hydroxyalkyl(meth)acrylates are not described.

EP-A 638 306 describes hair cosmetic VOC-80 preparations which, inter alia, comprise acrylic polymers or vinyl ester polymers. Copolymers which comprise copolymerized tert-butyl(meth)acrylates are not described.

EP-A 705 595 describes an acrylic hair-fixing resin which, based on the total weight of monomer, comprises from 5 to 95% by weight of at least one C1 to C8-alkyl(meth)acrylate monomer, from 2 to 70% by weight of at least one hydroxyalkyl(meth)acrylate monomer and from 2 to 50% by weight of at least one C1 to C8 monoethylenically unsaturated monocarboxylic acid monomer. Copolymers which comprise copolymerized tert-butyl(meth)acrylates are not described.

EP-A 605 686 describes aqueous emulsion polymers comprising polymerized radicals of (a) one or more ethylenically unsaturated acidic monomers, chosen from the group which consists of C3-C12-mono- and -dicarboxylic acids and the C1-C8-alkyl half-esters of maleic acid and fumaric acid and combinations thereof, present in an amount of from 5 to 35% by weight of the polymer, and (b) one or more water-insoluble comonomers, chosen from the group which consists of C3-C12-acrylates and -methacrylates, C1-C8-alkyl-substituted acrylamides and methacrylamides, vinyl esters of C3-C12-carboxylic acids, styrene and combinations thereof, present in an amount of from 65 to 95% by weight of the polymer, and (c) optionally one or more nonionic water-soluble comonomers chosen from one or more of the group which consists of water-soluble hydroxyalkyl esters of acrylic acid and methacrylic acid, C1-C4-alkyl-C2-C4-aminoalkyl esters of acrylic acid and methacrylic acid, acrylamide and methacrylamide, dimethylacrylamide and -methacrylamide, N-vinylpyrrolidone and vinyl-caprolactam, present in an amount of up to 20% by weight of the polymer. Copolymers which comprise copolymerized tert-butyl(meth)acrylates are not described.

EP-A 985 401 describes hair-setting polymers of (i) 5 to 95% by weight of C1-C12-alkyl(meth)acrylate, (ii) 2 to 70% by weight of hydroxyalkyl(meth)acrylate and (iii) 2 to 50% by weight of C3-C8 monoethylenically unsaturated monocarboxylic acid. Copolymers which comprise copolymerized tert-butyl(meth)acrylates are not described.

EP-A 985 405 and U.S. Pat. No. 6,214,328 describe hair-setting polymers of (i) 5 to 95% by weight of C1-C10-alkyl(meth)acrylate, (ii) 0 to 70% by weight of hydroxyalkyl(meth)acrylate and (iii) 0 to 50% by weight of C3-C8 monoethylenically unsaturated monocarboxylic acid and (iv) 1-25% by weight of monoethylenically unsaturated C4-C8-dicarboxylic acid. Copolymers which comprise copolymerized tert-butyl(meth)acrylates are not described.

EP-A 1 321 130 describes hair cosmetic preparations which comprise at least one (meth)acrylate copolymer, which comprises, in copolymerized form, (a) butyl(meth)acrylate, (b) hydroxyalkyl(meth)acrylate, (c) if appropriate, further monomers. Copolymers, which comprise both tert-butyl(meth)acrylate and methacrylic acid in copolymerized form are not described.

U.S. Pat. No. 3,577,518 describes alcoholic cosmetic preparations which comprise copolymers of hydroxyalkyl(meth)acrylates, alkyl(meth)acrylates and, if appropriate, cationic monomers. Copolymers which comprise copolymerized tert-butyl(meth)acrylates are not described.

U.S. Pat. No. 4,196,190 describes hair-setting preparations which copolymers of 10-30% by weight of alkyl acrylates, 41-60% by weight of methyl methacrylate, 5.20% by weight of hydroxyethyl methacrylate and 12-30% by weight of methacrylic acid. Copolymers which comprise copolymerized tert-butyl(meth)acrylates are not described.

U.S. Pat. No. 5,589,157 describes aqueous compositions which comprises copolymers of C1-C5-alkyl acrylate, C1-C5-alkyl methacrylate and C3-C5 ethylenically unsaturated carboxylic acid. Copolymers which comprise copolymerized tert-butyl(meth)acrylates are not described.

WO 02/38638 describes acrylate polymers with a K-value of 10-60 obtainable by free-radical polymerization of 30-99% by weight of tert-butyl acrylate and tert-butyl methacrylate as monomer A, 1-28% by weight of acrylic acid and/or methacrylic acid as monomer B and 0-60% by weight of a free-radically copolymerizable monomer and a free-radically copolymerizable monomer mixture as monomer C, where at least one of the monomers C provides a homopolymer with a glass transition temperature of less than 30° C. Copolymers, which comprise copolymerized hydroxyalkyl(meth)acrylates are not described.

### OBJECT AND SOLUTION

One object of the present invention was to provide polymers for cosmetic, in particular hair cosmetic, preparations which can be readily formulated as a clear liquid as pump or aerosol spray in solvents or solvent mixtures with an increased water fraction, whose formulations are readily sprayable in the form of small uniform droplets and, during and after application, have the lowest possible tendency for foaming and whose films then formed are not sticky and have good mechanical properties.

Besides the good compatibility with the customary cosmetic ingredients, the polymers applied to the hair should dry rapidly and impart good setting and prolonged hold to the hair even at increased atmospheric humidity, have a good ability to be washed out and be able to be formulated as optically clear VOC-55 aerosols (i.e. with a VOC fraction of at most 55% by weight). In addition, the treated hair should have good haptic properties such as, for example, a good feel to the touch.

Surprisingly, these objects were achieved by copolymers which comprise, in copolymerized form,
a) between 25 and 80% by weight of tert-butyl(meth)acrylate,
b) 2 to 60% by weight of hydroxyalkyl(meth)acrylate,
c) 10 to 40% by weight of an anionic or anionogenic, free-radically polymerizable compound which is or comprises methacrylic acid and
d) 0 to 30% by weight of at least one further free-radically polymerizable compound, where the amounts of the components a) to d) add up to 100% by weight.

Within the scope of the present invention, the expression alkyl comprises straight-chain and branched alkyl groups. Suitable short-chain alkyl groups are, for example, straight-chain or branched C₁-C₁₂-alkyl, preferably C₁-C₈-alkyl and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc. Suitable longer-chain C₈-C₃₀-alkyl or C₈-C₃₀-alkenyl groups are straight-chain and branched alkyl or alkenyl groups. These are preferably predominantly linear alkyl radicals as also arise in natural or synthetic fatty acids and fatty alcohols and also oxo alcohols, which may, if appropriate, additionally be mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene) etc.

Cycloalkyl is preferably C₅-C₈-cycloalkyl, such as cyclopentyl, cyclohexyl, cycloheptyl or cyclooctyl.

For the purposes of the present invention, the expression heterocycloalkyl comprises saturated, cycloaliphatic groups having generally 4 to 7, preferably 5 or 6, ring atoms, in which 1 or 2 of the ring carbon atoms are replaced by heteroatoms chosen from the elements oxygen, nitrogen and sulfur and which may, if appropriate, be substituted, where in the case of a substitution, these heterocycloaliphatic groups can carry 1, 2 or 3, preferably 1 or 2, particularly preferably 1, substituent chosen from alkyl, aryl, COOR, COO⁻M⁺ and NE¹E², preferably alkyl. Examples of such heterocycloaliphatic groups which may be mentioned are pyrrolidinyl, piperidinyl, 2,2,6,6-tetramethyl-piperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

Aryl comprises unsubstituted and substituted aryl groups and is preferably phenyl, tolyl, xylyl, mesityl, naphthyl, fluorenyl, anthracenyl, phenanthrenyl, naphthacenyl and, in particular, phenyl, tolyl, xylyl or mesityl.

Substituted aryl radicals preferably have 1, 2, 3, 4 or 5, in particular 1, 2 or 3, substituents chosen from alkyl, alkoxy, carboxyl, carboxylate, trifluoromethyl, —SO₃H, sulfonate, NE¹E², alkylene-NE¹E², nitro, cyano or halogen.

Hetaryl is preferably pyrrolyl, pyrazolyl, imidazolyl, indolyl, carbazolyl, pyridyl, quinolinyl, acridinyl, pyridazinyl, pyrimidinyl or pyrazinyl.

Arylalkyl is groups which comprise both alkyl and aryl radicals, these arylalkyl groups being joined to the compound carrying them either via the aryl radical or via the alkyl radical.

### Component a)

Preferably, the copolymer according to the invention comprises, based on the total amount of components a) to d), more than 30, particularly preferably at least 40, very particularly preferably at least 45 and in particular at least 50 and at most 75, particularly preferably at most 70, very particularly preferably at most 65 and in particular at most 60% by weight of copolymerized tert-butyl(meth)acrylate a).

Tert-butyl(meth)acrylate means tert-butyl methacrylate and/or tert-butyl acrylate.

Preferably, component a) is tert-butyl acrylate or a mixture of tert-butyl methacrylate and tert-butyl acrylate. If a mixture of tert-butyl methacrylate and tert-butyl acrylate is used as component, then the weight ratio of tert-butyl methacrylate to tert-butyl acrylate is preferably at least 1:1.5, particularly preferably at least 1:2 and in particular at least 1:2.5.

### Component b)

The copolymer according to the invention comprises, based on the total amount of components a) to d), at least 2, preferably at least 5, particularly preferably at least 10 and at most 60, preferably at most 40 and particularly preferably at most 35% by weight of copolymerized hydroxyalkyl(meth)acrylate b). Hydroxyalkyl(meth)acrylates are esters of (meth)acrylic acid with alkanes which carry at least 2 hydroxy groups, in particular with alkanediols.

Suitable hydroxyalkyl(meth)acrylates b) are, for example, 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate, 3-hydroxy-2-ethylhexyl methacrylate, neopentyl glycol mono(meth)acrylate, 1,5-pentanediol mono(meth)acrylate, 1,6-hexanediol mono(meth)acrylate and mixtures thereof.

Component b) is particularly preferably chosen from the group consisting of hydroxyethyl(meth)acrylate, 2-hydroxypropyl(meth)acrylate, 3-hydroxypropyl(meth)acrylate.

In particular component b) is chosen from the group consisting of hydroxyethyl methacrylate, 2-hydroxypropyl methacrylate, 3-hydroxypropyl methacrylate and mixtures thereof.

Very particularly preferred as component b) is hydroxyethyl methacrylate.

### Component c)

The copolymers according to the invention comprise, based on the total weight of components a) to d), 10 to 40% by weight of an anionic or anionogenic, free-radically polymerizable compound c), which is or comprises methacrylic acid, in copolymerized form.

An anionogenic compound is a compound which can be converted to the corresponding anionic form by deprotonation with bases.

Apart from methacrylic acid, component c) can comprise further anionic or anionogenic, free-radically polymerizable compounds. These are preferably chosen from the group consisting of acrylic acid, ethacrylic acid, α-chloroacrylic acid, crotonic acid, maleic acid, maleic anhydride, itaconic acid, citraconic acid, mesaconic acid, glutaconic acid, aconitic acid, fumaric acid and mixtures thereof. Apart from methacrylic acid, component c) can also comprise half-esters of monoethylenically unsaturated dicarboxylic acids having 4 to 10, preferably 4 to 6, carbon atoms, e.g. of maleic acid such as monomethyl maleate.

Apart from methacrylic acid, if component c) comprises further anionic or anionogenic, free-radically polymerizable compounds, then these are preferably chosen from the group consisting of acrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, particularly preferably acrylic acid.

Apart from methacrylic acid, if component c) also comprises further anionic or anionogenic, free-radically polymerizable compounds, then the weight ratio of methacrylic acid to these compounds, such as for example acrylic acid, is preferably at least 2:1, particularly preferably at least 2.5:1 and in particular at least 3:1.

Components c) are also the salts of the abovementioned acids, in particular the sodium, potassium and ammonium salts. The stated weight fractions refer to the acid form.

A preferred subject-matter of the invention is a copolymer according to the invention where component c) is methacrylic acid or a mixture of methacrylic acid and at least one further monoethylenically unsaturated carboxylic acid. In particular, component c) is methacrylic acid or a mixture of methacrylic acid and acrylic acid.

To prepare the copolymers, component c) can be used in partially or completely deprotonated form. Their counterions are then preferably derived from bases as described below for adjusting the pH during the polymerization or of the polymers obtained. Preference is given to cosmetically acceptable salts, such as, for example alkali metal salts (e.g. sodium, potassium) or ammonium salts.

Preferably, the copolymer according to the invention comprises, based on the total amount of components a) to d), at least 12, particularly preferably at least 15 and at most 35, particularly preferably at most 30% by weight of component c) in copolymerized form.

### Component d)

If appropriate, the copolymer according to the invention comprises, based on the total amount of components a) to d), 0 to 30% by weight of at least one further free-radically polymerizable compound d) in copolymerized form.

Preferably the amount of this copolymerized component d), based on the total amount of components a) to d), is 0-20, particularly preferably 0-10 and very particularly preferably 0.5-5% by weight.

Suitable as component d) are all free-radically polymerizable compounds different from components a) to c) which can be copolymerized with components a) to c).

Preferred components d) different from components a), b) and c) are given below.

d1) essentially hydrophobic, nonionic compounds, preferably esters of vinyl alcohol or allyl alcohol with C1-C30-monocarboxylic acids, vinyl ethers, vinyl aromatics, vinyl halides, vinylidene halides, C2-C8-monoolefins, nonaromatic hydrocarbons having at least 2 conjugated double bonds and mixtures thereof.

Suitable compounds d1) are, accordingly, for example vinyl formate, vinyl acetate, vinyl propionate, vinyl-n-butyrate, vinyl stearate, vinyl laurate, styrene, α-methylstyrene, o-chlorostyrene, acrylonitrile, methacrylonitrile, vinyltoluenes, vinyl chloride, vinylidene chloride, vinyl fluoride, vinylidene fluoride, ethylene, propylene, isobutene, butadiene, isoprene, chloroprene, methyl, ethyl, butyl, dodecyl vinyl ethers and mixtures thereof. Preferred compounds d1) are also the esters of α,β-ethylenically unsaturated mono- and dicarboxylic acids with C1-C30 alkanols, preferably C1-C22 alkanols, different from a). Preference is also given to the amides of α,β-ethylenically unsaturated mono- and dicarboxylic acids with mono- and dialkylamines, which have 1 to 30 carbon atoms, preferably 1 to 22 carbon atoms per alkyl radical.

These are preferably compounds of the general formula III

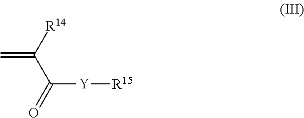

in which
R¹⁴ is hydrogen or C1- to C8-alkyl,
R¹⁵ is a straight-chain or branched C1- to C30-alkyl radical, and
Y is O or NR¹⁶, where R¹⁶ is hydrogen, C1- to C8-alkyl or C5- to C8-cycloalkyl.

Preferably, in the formula III R¹⁴ is hydrogen, methyl or ethyl.

Preferably, Y is O or NH.

Suitable radicals R¹⁵ are the abovementioned C1-C30-alkyl radicals. In particular, R¹⁵ is methyl, ethyl, n-propyl, isopropyl, n-butyl, 2-butyl, sec-butyl, tert-butyl, n-pentyl, n-hexyl, n-heptyl, n-octyl, ethylhexyl, 1,1,3,3-tetramethylbutyl, undecyl, lauryl, tridecyl, myristyl, pentadecyl, palmityl, margarinyl, stearyl, palmitoleinyl, oleyl or linolyl.

In particular component d1) is chosen from methyl(meth)acrylate, methyl ethacrylate, ethyl(meth)acrylate, n-propyl(meth)acrylate, i-propyl(meth)acrylate, n-butyl(meth)acrylate, 1-butyl(meth)acrylate, sec-butyl(meth)acrylate, 2-pentyl(meth)acrylate, 3-pentyl(meth)acrylate, isopentyl(meth)acrylate, neopentyl(meth)acrylate, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arrachinyl(meth)acrylate, behenyl(meth)acrylate, lignocerenyl(meth)acrylate, cerotinyl(meth)acrylate, melissinyl(meth)acrylate, palmitoleinyl(meth)acrylate, oleyl(meth)acrylate, linolyl(meth)acrylate, lignolenyl(meth)acrylate, stearyl(meth)acrylate, lauryl(meth)acrylate, phenoxyethyl(meth)acrylate, 4-t-butylcyclohexyl acrylate, cyclohexyl(meth)acrylate, ureido(meth)acrylate, tetrahydrofurfuryl(meth)acrylate and mixtures thereof.

Component d1) can also be chosen from (meth)acrylamide, N-methyl(meth)acrylamide, N-ethyl(meth)acrylamide, N-n-propyl(meth)acrylamide, N-i-propyl(meth)acrylamide, N-(n-butyl)methacrylamide, N-(sec-butyl)methacrylamide, N-(tert-butyl)methacrylamide, N-(n-pentyl)(meth)acrylamide, N-(n-hexyl)(meth)acrylamide, N-(n-heptyl)(meth)acrylamide, tert-butyl(meth)acrylamide, n-octyl(meth)acrylamide, 1,1,3,3-tetramethylbutyl(meth)acrylamide, ethylhexyl(meth)acrylamide, n-nonyl(meth)acrylamide, n-decyl(meth)acrylamide, n-undecyl(meth)acrylamide, tridecyl(meth)acrylamide, myristyl(meth)acrylamide, pentadecyl(meth)acrylamide, palmityl(meth)acrylamide, heptadecyl(meth)acrylamide, nonadecyl(meth)acrylamide, arrachinyl(meth)acrylamide, behenyl(meth)acrylamide, lignocerenyl(meth)acrylamide, cerotinyl(meth)acrylamide, melissinyl(meth)acrylamide, palmitoleinyl(meth)acrylamide, oleyl(meth)acrylamide, linolyl(meth)acrylamide, linolenyl(meth)acrylamide, stearyl(meth)acrylamide, lauryl(meth)acrylamide and mixtures thereof.

d2) Compounds with at least one ionogenic or ionic group per molecule

The compounds d2) have at least one ionogenic or ionic group per molecule which is preferably chosen from carboxylate groups and/or sulfonate groups and salts thereof obtainable by partial or complete neutralization with a base, and also tertiary amino groups, which may be partially or completely protonated and quaternized. Suitable bases for the neutralization and acids for the protonation and alkylating agents for the quaternization are known to the person skilled in the art.

Suitable compounds d2) are acrylamidoalkanesulfonic acids and salts thereof, such as 2-acrylamido-2-methylpropanesulfonic acid and alkali metal salts thereof, e.g. sodium and potassium salts thereof.

Further suitable compounds d2) are the esters of the abovementioned, α,β-ethylenicaily unsaturated mono- and dicarboxylic acids with C2- to C12-amino alcohols, which are C1- to C8-dialkylated on the amine nitrogen. These include, for example N,N-dimethylaminomethyl(meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate, N,N-dimethylaminocyclohexyl(meth)acrylate etc. Preference is given to using N,N-dimethylaminopropyl acrylate and N,N-dimethylaminopropyl(meth)acrylate.

Suitable compounds d2) are also the amides of the abovementioned α,β-ethylenically unsaturated mono- and dicarboxylic acids with diamines which have a tertiary and a primary or secondary amino group. These include, for example, N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide or N-[4-(dimethylamino)cyclohexyl]methacrylamide.

Suitable compounds d2) are also vinyl- and allyl-substituted heteroaromatic compounds, such as 2- and 4-vinylpyridine, allylpyridine, and preferably N-vinyl heteroaromatics, such as N-vinylimidazole or N-vinyl-2-methylimidazole.

d3) essentially hydrophilic, nonionic compounds, preferably N-vinylamides, N-vinyllactams, primary amides of α,β-ethylenically unsaturated monocarboxylic acids, vinyl- and allyl-substituted heteroaromatic compounds, esters of α,β-ethylenically unsaturated mono- and dicarboxylic acids with C1-C30-alkanediols, different from b), esters and amides of α,β-ethylenically unsaturated mono- and dicarboxylic acids with C2-C30-amino alcohols, which have a primary or secondary amino group, polyether acrylates and mixtures thereof.

Suitable N-vinyllactams d3) are, for example those which have one or more C1-C6-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam.

Suitable compounds d3) are also primary amides of the abovementioned α,β-ethylenically unsaturated monocarboxylic acids, such as acrylamide, methacrylamide and ethacrylamide.

Suitable compounds d3) are also the esters of the abovementioned acids with triols and polyols, such as glycerol, erythritol, pentaerythritol or sorbitol. Suitable compounds d3) are also polyether acrylates.

Suitable components e5) and e6) are also N-vinylimidazoles of the general formula VII, in which R¹ to R³ is hydrogen, C₁-C₄-alkyl or phenyl

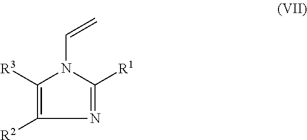

Examples of compounds of the general formula VII are given in Table 1 below:

TABLE 1

| R¹: | R²: | R³: |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl; Ph = phenyl

The charged cationic groups can be produced from the amine nitrogens by quaternization with so-called alkylating agents. Examples of suitable alkylating agents are C₁-C₄-alkyl halides or sulfates, such as ethyl chloride, ethyl bromide, methyl chloride, methyl bromide, dimethyl sulfate and diethyl sulfate. A quaternization can generally take place either before or after the polymerization.

d4) compounds with at least two free-radically polymerizable double bonds

Suitable as component d) are also compounds with at least two free-radically polymerizable, olefinically unsaturated double bonds per molecule. Such compounds are usually referred to as crosslinkers.

Free-radically polymerizable, olefinically unsaturated double bonds are, for example, alkenyl groups, which formally arise by detaching a H atom from an alkene. These include vinyl (—CH═CH₂), 1-propenyl (—CH═CH—CH₃), 2-propenyl or allyl (—CH₂—CH═CH₂), 1-butenyl (—CH═CH—CH₂—CH₃) etc.

Alkylidene groups, i.e. groups which are joined to a carbon atom of a molecule by a double bond, also belong to the free-radically polymerizable, olefinically unsaturated double bonds (example ethylidene: ═CHCH₃).

Suitable crosslinkers are, for example, acrylic esters, methacrylic esters, allyl ethers or vinyl ethers of at least dihydric alcohols. The OH groups of the parent alcohols can here by completely or partially etherified or esterified; however, the crosslinkers comprise at least two olefinically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000.

Apart from the homopolymers of ethylene oxide and propylene oxide, it is also possible to use block copolymers of ethylene oxide or propylene oxide or copolymers which comprise ethylene oxide and propylene oxide groups in incorporated form.

Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates. The polyhydric alcohols can also firstly be converted to the corresponding glycidyl ethers by reaction with epichlorohydrin.

Further suitable crosslinkers are the vinyl esters or the esters of monohydric, unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is, however, also possible to esterify the monohydric, unsaturated alcohol with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the polyhydric alcohols described above, for example oleic acid, crotonic acid, cinnamic acid or 10-undecenoic acid.

Suitable crosslinkers are, furthermore, straight-chain or branched, linear or cyclic aliphatic or aromatic hydrocarbons which have at least two double bonds which in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Further suitable crosslinkers are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids such as acrylic acid, methacrylic acid, itaconic acid, maleic acid or at least dibasic carboxylic acids, as have been described above.

In addition, triallylamine and triallylmonoalkylammonium salts, e.g. triallylmethylammonium chloride or methyl sulfate, are suitable as crosslinkers.

Also suitable are N-vinyl compounds of urea derivatives, at least difunctional amides, cyanurates or urethanes, for example of urea, ethylene urea, propylene urea or tartaramide, e.g. N,N′-divinylethyleneurea or N,N′-divinylpropyleneurea.

Also suitable are alkylenebisacrylamides, such as methylenebisacrylamide and N,N″-(2,2)-butane and 1,1′-bis(3,3′-vinylbenzimidazolith-2-one)-1,4-butane.

Other suitable crosslinkers are, for example, alkylene glycol di(meth)acrylates such as ethylene glycol diacrylate, ethylene glycol dimethacrylate, tetraethylene glycol acrylate, tetraethylene glycol dimethacrylate, diethylene glycol acrylate, diethylene glycol methacrylate, vinyl acrylate, allyl acrylate, allyl methacrylate, divinyldioxane, pentaerythritol allyl ether, and mixtures of these crosslinkers.

Further suitable crosslinkers are divinyldioxane, tetraallylsilane or tetravinylsilane.

Crosslinkers which are particularly preferably used are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols, which have been reacted with ethylene oxide and/or propylene oxide and/or epichlorohydrin.

Very particularly preferred crosslinkers are allyl methacrylate, pentaerythritol triallyl ether, methylenebisacrylamide, N,N′-divinylethyleneurea, triallylamine and triallylmonoalkylammonium salts, and acrylic esters of glycol, butanediol, trimethylolpropane or glycerol or acrylic esters of glycol, butanediol, trimethylolpropane or glycerol reacted with ethylene oxide and/or epichlorohydrin.

Mixtures of the abovementioned compounds can of course also be used. The crosslinker is preferably soluble in the reaction medium. If the solubility of the crosslinker in the reaction medium is slight, then it can be dissolved in a monomer or in a monomer mixture, or else be metered in dissolved in a solvent which is miscible with the reaction medium. Particular preference is given to those crosslinkers which are soluble in the monomer mixture.

### Preparation of the Copolymers

The copolymers according to the invention are prepared by customary processes known to the person skilled in the art, e.g. by solution polymerization, precipitation polymerization, suspension polymerization or emulsion polymerization.

Preparation is preferably by solution polymerization or emulsion polymerization.

Preferred solvents for the solution polymerization are alcohols or mixtures of alcohols and water, such as methanol, ethanol, n-propanol, isopropanol, n-butanol, sec-butanol, tert-butanol, n-hexanol and cyclohexanol, and also glycols, such as ethylene glycol, propylene glycol and butylene glycol and also the methyl or ethyl ethers of dihydric alcohols, diethylene glycol, triethylene glycol, polyethylene glycols with number-average molecular weights up to about 3000, glycerol and dioxane.

The polymerization is particularly preferably in alcohol or a water/alcohol mixture, for example in a water/ethanol mixture. If water is used as solvent constituent, preference is given to using demineralized water.

The copolymers according to the invention are particularly preferably prepared in a known manner by free-radically initiated aqueous emulsion polymerization of the monomers a), b) and, if appropriate, c).

### Emulsion Polymerization

The method of free-radically initiated aqueous emulsion polymerization has already been described many times and is therefore sufficiently known to the person skilled in the art [cf. e.g. Encyclopedia of Polymer Science and Engineering, Vol. 8, pages 659 to 677, John Wiley & Sons, Inc., 1987; D. C. Blackley, Emulsion Polymerisation, pages 155 to 465, Applied Science Publishers, Ltd., Essex, 1975; D. C. Blackley, Polymer Latices, 2nd edition, Vol. 1, pages 33 to 415, Chapman & Hall, 1997; H. Warson, The Applications of Synthetic Resin Emulsions, pages 49 to 244, Ernest Benn, Ltd., London, 1972; D. Diederich, Chemie in unserer Zeit [Chemistry in our Time] 1990, 24, pages 135 to 142, Verlag Chemie, Weinheim; J. Piirma, Emulsion Polymerisation, pages 1 to 287, Academic Press, 1982; F. Holscher, Dispersionen synthetischer Hochpolymerer [Dispersions of synthetic high polymers], pages 1 to 160, Springer-Verlag, Berlin, 1969 and DE-A 40 03 422].

The free-radically initiated aqueous emulsion polymerization usually takes place by dispersely distributing the monomers, generally in the presence of dispersants, in aqueous medium, and polymerizing them by means of at least one free-radical polymerization initiator.

### Initiators

Suitable free-radical polymerization initiators are always initiators which are able to trigger a free-radical aqueous emulsion polymerization.

These may in principle be peroxides or azo compounds. These may be inorganic or organic in nature. Redox initiator systems are of course also suitable.

Peroxides which can be used are in principle inorganic peroxides, such as hydrogen peroxide or peroxodisulfates, such as the mono- or dialkali metal or ammonium salts of peroxodisulfuric acid, such as, for example, its mono- and disodium, -potassium or ammonium salts or organic peroxides, such as alkyl hydroperoxides, for example tert-butyl, p-menthyl or cumyl hydroperoxide, tert-butyl perpivalate, and dialkyl or diaryl peroxides, such as di-tert-butyl peroxide or dicumyl peroxide, 2,5-dimethyl-2,5-di(t)butylperoxy(hexane) or dibenzoyl peroxide.

As azo compound, essentially 2,2″-azobis(isobutyronitrile), 2,2′-azobis(2,4-dimethylvaleronitrile) and 2,2′-azobis(amidinopropyl)dihydrochloride (AIBA, corresponds to V-50TM from Wako Chemicals), 1,1′-azobis(1-cyclohexanecarbanitrile), 2,2′-azobis(2-amidinopropane) salts, 4,4′-azobis(4-cyanovaleric acid) or 2-(carbamoylazo)isobutyronitrile are used.

Suitable oxidizing agents for redox initiator systems are essentially the peroxides given above. Corresponding reducing agents which can be used are sulfur compounds with a low oxidation state, such as alkali metal sulfites, for example potassium and/or sodium sulfite, alkali metal hydrogensulfites, for example potassium and/or sodium hydrogensulfite, alkali metal metabisulfites, for example potassium and/or sodium metabisulfite, formaldehyde sulfoxylates, for example potassium and/or sodium formaldehyde sulfoxylate, alkali metal salts, specifically potassium and/or sodium salts of aliphatic sulfinic acids and alkali metal hydrogensulfides, such as, for example, potassium and/or sodium hydrogensulfide, salts of polyvalent metals, such as iron (II) sulfate, iron (II) ammonium sulfate, iron (II) phosphate, enediols, such as dihydroxymaleic acid, benzoin and/or ascorbic acid, and reducing saccharides, such as sorbose, glucose, fructose and/or dihydroxyacetone.

The initiators are usually used in amounts of from 0.001 to 10% by weight, preferably 0.02 to 5% by weight, based on the monomers to be polymerized.

### Surfactants and Protective Colloids

The emulsion polymerization is usually carried out in the presence of surfactants and/or protective colloids.

During the preparation of the copolymers according to the invention, at least one dispersion auxiliary is co-used which can keep both the monomer droplets and also polymer particles in disperse distribution in the aqueous phase, and thus ensures the stability of the aqueous polymer dispersion produced. Of suitability are either the protective colloids customarily used for carrying out free-radical aqueous emulsion polymerizations, or emulsifiers.

Suitable protective colloids are, for example polyvinyl alcohols, cellulose derivates or copolymers comprising vinyl pyrrolidone. A detailed description of further suitable protective colloids is given in Houben-Weyl, Methoden der organischen Chemie [Methods of organic chemistry], Volume XIV/1, Makromolekulare Stoffe [Macromolecular substances], pages 411 to 420, Georg-Thieme-Verlag, Stuttgart, 1961

Mixtures of emulsifiers and/or protective colloids can of course also be used. Preferably, the dispersion auxiliaries used are exclusively emulsifiers whose relative molecular weights are, in contrast to the protective colloids, usually below 1000. They can be anionic, cationic or nonionic in nature. When using mixtures of interface-active substances, the individual components must of course be compatible with one another, something which can be checked in cases of doubt using a few preliminary experiments. In general, anionic emulsifiers are compatible with one another and with nonionic emulsifiers. The same is also true for cationic emulsifiers, whereas anionic and cationic emulsifiers are in most cases not compatible with one another.

Customary emulsifiers are, for example, ethoxylated mono-, di- and trialkylphenols (degree of EO: 3 to 50, alkyl radical: C4 to C12), ethoxylated fatty alcohols (degree of EO: 3 to 50; alkyl radical: C8 to C36) and alkali metal and ammonium salts of alkyl sulfates (alkyl radical: C8 to C12), of sulfuric half-esters of ethoxylated alkanols (degree of EO: 4 to 30, alkyl radical: C12 to C18) and ethoxylated alkylphenols (degree of EO: 3 to 50, alkyl radical: C4 to C12), of alkylsulfonic acids (alkyl radical: C12 to C18) and of alkylarylsulfonic acids (alkyl radical: C9 to C18). Further suitable emulsifiers are given in Houben-Weyl, Methoden der organischen Chemie [Methods of organic chemistry], Volume XIV/1, Makromolekulare Stoffe [Macromolecular substances], pages 192 to 208, Georg-Thieme-Verlag, Stuttgart, 1961.

Proven interface-active substances are also compounds of the general formula II

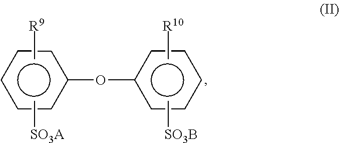

in which R⁹ and R¹⁰ are C4- to C24-alkyl and one of the radicals R⁹ or R¹⁰ can also be hydrogen, and A and B may be alkali metal ions and/or ammonium ions. In the general formula II R⁹ and R¹⁹ are preferably linear or branched alkyl radicals having 6 to 18 C atoms, in particular having 6, 12 and 16 C atoms or H atoms, where R⁹ and R¹⁹ are not both H atoms at the same time. A and B are preferably sodium, potassium or ammonium ions, with sodium ions being particularly preferred. Particularly advantageous compounds II are those in which A and B are sodium ions, R⁹ is a branched alkyl radical with 12 C atoms and R¹⁰ is an H atom or R⁹. Technical-grade mixtures are often used that have a fraction of from 50 to 90% by weight of the monoalkylated product, for example Dowfax®2A1 (Dow Chemical Company). The compounds II are generally known, e.g. from U.S. Pat. No. 4,269,749, and are commercially available.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and also ammonium and triethanolamine salts. The alkyl ether sulfates, alkyl ether phosphates, alkyl glycol alkoxylates and diglycol alkoxylates and alkyl ether carboxylates can have between 1 to 10 ethylene oxide or propylene oxide units, preferably 1 to 3 ethylene oxide units, in the molecule.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. Also suitable are alkylamine oxides, mono- or dialkylalkanolamides, fatty acid esters of polyethylene glycols, ethoxylated fatty acid amides, alkyl polyglycosides, alkyl glycol alkoxylates and diglycol alkoxylates or sorbitan ether esters.

Suitable surfactants are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

As a rule, the amount of dispersion auxiliary used is 0.1 to 5% by weight, preferably 1 to 3% by weight, in each case based on the total amount of the monomers to be free-radically polymerized. It is often favorable if some or all of the dispersion auxiliary is added to the fluid reaction medium before initiating the free-radical polymerization. Moreover, some or all of the dispersion auxiliary can be added to the reaction medium advantageously also together with the monomers to be polymerized, in particular in the form of an aqueous monomer emulsion during the polymerization.

Preference is given to a combination of ionic and nonionic dispersion auxiliaries.

### Regulators

Using so-called regulators it is possible to influence the molecular weight of polymers. The regulators used are preferably alkanethiols. It is also possible to use mixtures of two or more regulators.

The alkanethiols used are linear and branched alkanethiols with a carbon chain length of from C10 to C22. Particular preference is given to linear alkanethiols, preference also being given to alkanethiols with a chain length of from C12 to C22, in particular from C12 to C18. Preferred alkanethiols may which may be mentioned are n-decanethiol, n-dodecanethiol, tert-dodecanethiol, n-tetradecanethiol, n-pentadecanethiol, n-hexadecanethiol, n-heptadecanethiol, n-octadecanethiol, n-nonadecanethiol, n-eicosanethiol, n-docosanethiol. Particular preference is given to linear, even-numbered alkanethiols. The alkanethiols can also be used in mixtures. The alkanethiols are usually used in amounts of from 0.1 to 5% by weight, in particular 0.25 to 2% by weight, based on the monomers to be polymerized. Usually, the alkanethiols are added to the polymerization together with the monomers.

A particularly preferred regulator is n-dodecanethiol (n-dodecyl mercaptan).

### Hydrogen Peroxide Treatment

If alkanethiols are used during the polymerization, a subsequent hydrogen peroxide treatment is required, if appropriate, in order to obtain polymers with a neutral odor. For this hydrogen peroxide treatment, which follows the polymerization, usually 0.01 to 2.0% by weight, in particular 0.02 to 1.0% by weight, preferably 0.3 to 0.8% by weight, further preferably 0.03 to 0.15% by weight, of hydrogen peroxide are used, based on the amount of the monomers to be polymerized. It has proven advantageous to carry out the hydrogen peroxide treatment at a temperature of from 20 to 100° C., in particular from 30 to 80° C. The hydrogen peroxide treatment is usually carried out for a period from 15 min to 240 min, in particular from 30 min to 90 min.

If alkanethiols with a C chain length of from C14 to C22 are used the hydrogen peroxide treatment can be omitted. In a further embodiment of the invention, however, a hydrogen peroxide treatment can be subsequently carried out even when using alkanethiols with a chain length of from C14 to C22.

### Carrying Out the Emulsion Polymerization

The emulsion polymerization usually takes place with the exclusion of oxygen, for example under an atmosphere of nitrogen or argon at temperatures in the range of from 20 to 200° C. Polymerization temperatures in the range of from 50 to 130, in particular from 70 to 95° C., are advantageous.

In the case of free-radically initiated emulsion polymerization, it has to be ensured, especially at elevated temperatures, to avoid coagulation that the polymer mixture does not boil. This can be avoided, for example, by the polymerization reaction taking place at an inert-gas pressure which is higher than the vapor pressure of the polymerization mixture, for example 1.2 bar, 1.5 bar, 2 bar, 3 bar, 5 bar, 10 bar or even higher (in each case absolute values). The polymerization can be carried out batchwise, semicontinuously or continuously. The polymerization and/or the metered addition of monomer and regulator often takes place semicontinuously by the feed method.

The amounts of monomers and dispersants are expediently chosen to give a 30 to 80% by weight dispersion of the copolymers. Preferably, at least some of the monomers, initiators and, if appropriate, regulators are uniformly metered in through the reaction vessel (feed procedure) during the polymerization. The monomers and the initiator can, however, also be initially introduced in the reactor and polymerized, if appropriate with cooling.

According to a preferred embodiment, the polymerization is carried out using a seed latex. The seed latex is expediently prepared from the polymers to be polymerized in the first polymerization phase in the customary manner. The remaining monomer mixture is then added, preferably by the feed method.

The polymerization reaction advantageously takes place to a monomer conversion of at least 95% by weight, preferably at least 98% by weight, particularly preferably at least 99% by weight.

It is often useful if the aqueous polymer dispersion obtained is subjected to an after-polymerization step to further reduce the amount of unreacted monomer. This measure is known to the person skilled in the art (for example EP-B 3957, EP-B 28 348, EP-B 563 726, EP-A 764 699, EP-A 76 180, DE-A 3718 520, DE-A 3834734, DE-A 4232194, DE-A 19529599, DE-A 19741187, DE-A 19839199, DE-A 19840586, WO 95/33775 or U.S. Pat. No. 4,529,753).

### Preparation of the Dispersions

If the copolymer is prepared by emulsion polymerization, the dispersion obtained can either be incorporated directly into aqueous, aqueous-alcoholic or alcoholic hair cosmetic preparations or the dispersion is dried, e.g. by spray-drying, fluidized spray drying, drum drying or freeze-drying, so that the copolymer can be used and processed as powder.

Spray-drying is preferably used as a drying method. The dry polymer powders obtained in this way can advantageously be converted again into an aqueous solution or dispersion by dissolution or redispersion, respectively, in water.

Pulverulent copolymers have the advantage of better storage properties, easier transportation and generally have a lower tendency for microbial attack.

It is of course also possible to subject the aqueous polymer dispersion to an inert-gas and/or water-vapor stripping, which are likewise known to the person skilled in the art, before or after the after-polymerization step. This stripping operation preferably takes place after the after-polymerization step. As described in EP-A 805 169, partial neutralization of the dispersion to a pH in the range from 5 to 7, preferably to a pH in the range from 5.5 to 6.5, before physical deodorization is advantageous.

For the partial neutralization to pH 5-7, it is also possible to use all of the bases specified below under “Neutralization”.

### Neutralization

For certain uses, it is advantageous if the copolymers are at least partially neutralized. Partial or complete neutralization of the copolymer dispersions is advantageous especially for using the copolymers in (hair) cosmetic preparations.

Alkali metal or ammonium carbonate or hydrogen carbonate are suitable for the neutralizations.

The neutralization can also take place with
a mono-, di- or trialkanolamine having 2 to 5 carbon atoms in the alkanol radical, which, if appropriate, is present in etherified form, for example mono-, di- and triethanolamine, mono-, di- and tri-n-propanolamine, mono-, di- and triisopropanolamine, 2-amino-2-methylpropanol and di(2-methoxyethyl)amine,
an alkanediolamine having 2 to 5 carbon atoms, for example 2-amino-2-methylpropane-1,3-diol and 2-amino-2-ethylpropane-1,3-diol, or
a primary, secondary or tertiary alkylamine having a total of 5 to 10 carbon atoms, for example N,N-diethylpropylamine or 3-diethylamino-1-propylamine.

Suitable alkali metal hydroxides for the neutralization are primarily sodium hydroxide, or potassium hydroxide and ammonium hydroxide.

Good neutralization results are often obtained with 2-amino-2-methylpropanol, triisopropanolamine, 2-amino-2-ethylpropane-1,3-diol, N,N-dimethylaminoethanol or 3-diethylamino-1-propylamine.

Of suitability for neutralizing the polymers in the preparations and compositions according to the invention are, in particular, also silicone polymers comprising amino groups. Suitable silicone polymers comprising amino groups are, for example, the silicone-amino polyalkylene oxide block copolymers of WO 97/32917, the products SilsoftÒA-843 (Dimethicone Bisamino Hydroxypropyl Copolyol) and SilsoftÒA-858 (Trimethylsilyl Amodimethicone Copolymer) (both Witco). In addition, the neutralization polymers of EP-A 1035144 and, in particular, the silicone-containing neutralization polymers of claim 12 of EP-A 1035144 are also suitable.

In a preferred embodiment, the copolymers are, for example, at least 10, preferably at least 30, further preferably at least 40, particularly preferably at least 50, very particularly preferably at least 70 and in particular at least 95% neutralized.

In a particularly preferred embodiment, the polymers are at least 99% neutralized. Most preferably, the neutralization is at least 100%.

It is also possible for the neutralizing agent to be added in more than an equivalent amount, equivalent amount being understood as meaning the amount which is required in order to neutralize all neutralizable groups of the copolymer.

### Preservation of the Dispersions

Customary preservatives are used for stabilizing and preserving the dispersion polymers. Preference is given to using hydrogen peroxide.

### K Value

The K value of the copolymers according to the invention is in the range from 15 to 120, preferably from 25 to 75 and particularly preferably from 25 to 55. Most preferably, K values are in the range of from 25 to 45 (determination in accordance with Fikentscher, Cellulosechemie, Vol. 13, pp. 58 to 64 (1932)).

Ways of adjusting the K value of the copolymers to a desired value are known to the person skilled in the art. For example, these are the polymerization temperature, the amount of initiator or the use of chain-transfer reagents (regulators).

### Cosmetic Preparations

The term VOC is known to the person skilled in the art. VOC (volatile organic compounds) are organic chemical compounds which boil at atmospheric pressure in a range up to about 260° C. and can thus enter the atmosphere in gaseous form. Volatile organic compounds include numerous solvents and propellants.

Classification of organic compounds inside
(according to: WHO—World Health Organization 1989)

| | | Boiling point range |
|---|---|---|
| Compounds | Abbreviation | [° Celsius] |
| Very volatile organic compounds | VVOC | <0 to 50° C. |
| Volatile organic compounds | VOC | 50 to 260° C. |
| Semi volatile organic compounds | SVOC | 260 to 380° C. |

The copolymers according to the invention are exceptionally suitable for preparing cosmetic, in particular skin and/or hair cosmetic, preparations. They are used here, for example, as polymeric film formers. They can be used and formulated universally in a very wide variety of cosmetic, preferably hair cosmetic, preparations and are compatible with the usual components.

The copolymers according to the invention are advantageously suitable for producing elastic hair styles coupled with strong hold, even at high atmospheric humidity. The copolymers according to the invention are characterized by good propellant gas compatibility, good solubility in aqueous/alcoholic solvent mixtures, in particular by the suitability for use as optically clear low-VOC formulations and by good ability to be washed out and combed out without flaking effect. In addition, they improve hair treated with them in its sensorally perceptible properties, such as feel, volume or handlability. Hairspray formulations based on the copolymers according to the invention are characterized by good sprayability and good rheological properties and extremely low stickiness of the resulting films. The cosmetic, preferably hair cosmetic, preparations according to the invention comprising the copolymers according to the invention do not tend to foam following application. Besides the good compatibility with customary cosmetic ingredients, the applied copolymers according to the invention dry rapidly.

Accordingly, the present invention further provides the use of the copolymers according to the invention in cosmetic preparations, and also such cosmetic preparations per se.

### Cosmetically Acceptable Carrier B

The cosmetic preparations according to the invention are preferably aqueous preparations which comprise at least 10, preferably at least 20 and particularly preferably at least 30% by weight of water. Preferably, the cosmetic preparations according to the invention comprise at most 80, preferably at most 55% by weight of volatile organic constituents.

Accordingly, a particularly preferred embodiment covers cosmetic preparations according to the invention in which the fraction of volatile organic components is at most 55% by weight, based on the cosmetic preparation.

Besides water and the copolymers according to the invention, the cosmetic preparations according to the invention also have at least one cosmetically acceptable carrier B which is chosen from
i) water-miscible organic solvents, preferably C₂-C₄-alkanols, in particular ethanol,
ii) oils, fats, waxes,
iii) esters of C₆-C₃₀-monocarboxylic acids with mono-, di- or trihydric alcohols which are different from ii),
iv) saturated acyclic and cyclic hydrocarbons,
v) fatty acids,
vi) fatty alcohols,
vii) propellants (propellant gases) and
viii) mixtures thereof.

Suitable carriers B and further active ingredients and additives to be used advantageously are described in detail below.

Suitable cosmetically and pharmaceutically compatible oil and fat components are described in Karl-Heinz Schrader, Grundlagen und Rezepturen der Kosmetika, [Fundamentals and formulations of cosmetics], 2nd Edition, Verlag Hüthig, Heidelberg, pp. 319-355, which is hereby incorporated by reference.

The preparations according to the invention can, for example, have an oil or fat component as cosmetically acceptable carrier B which is chosen from: hydrocarbons of low polarity, such as mineral oils; linear saturated hydrocarbons, preferably having more than 8 carbon atoms, such as tetradecane, hexadecane, octadecane, etc.; cyclic hydrocarbons, such as decahydronaphthalene; branched hydrocarbons; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof. Suitable silicone oils B) are, for example, linear polydimethylsiloxanes, poly(methyl-phenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil and fat components B) are chosen from paraffin and paraffin oils; vaseline; natural fats and oils, such as castor oil, soybean oil, peanut oil, olive oil, sunflower oil, sesame oil, avocado oil, cocoa butter, almond oil, persic oil, ricinus oil, cod liver oil, lard, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candelilla wax, spermaceti and mixtures of the abovementioned oil and fat components.

Suitable hydrophilic carriers B) are chosen from water, 1-, 2- or polyhydric alcohols having preferably 1 to 8 carbon atoms, such as ethanol, n-propanol, isopropanol, propylene glycol, glycerol, sorbitol, etc.

The cosmetic preparations according to the invention may be skin cosmetic, hair cosmetic, dermatological, hygiene or pharmaceutical compositions. On account of their film-forming and flexible properties, the copolymers according to the invention described above are particularly suitable as additives for hair and skin cosmetics.

Preferably, the preparations according to the invention which comprise the copolymers according to the invention are in the form of a spray, gel, foam, ointment, cream, emulsion, suspension, lotion, milk or paste. If desired, liposomes or microspheres can also be used.

Preferably, the cosmetic compositions according to the invention comprise at least one copolymer according to the invention, at least one carrier B as defined above and at least one constituent different therefrom which is preferably chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tints, tanning agents, dyes, pigments, consistency regulators, humectants, refatting agents, collagen, protein hydrolysates, lipids, antioxidants, antifoams, antistats, emollients and softeners.

The preparations according to the invention preferably have a pH of from 2.0 to 9.3. The pH range is particularly preferably between 4 and 8. Organic solvents or a mixture of solvents with a boiling point below 400° C. may be present as additional cosolvents in an amount of from 0.1 to 15% by weight, preferably from 1 to 10% by weight. Particularly suitable additional cosolvents are unbranched or branched hydrocarbons, such as pentane, hexane, isopentane and cyclic hydrocarbons, such as cyclopentane and cyclohexane. Further particularly preferred water-soluble solvents are glycerol, ethylene glycol and propylene glycol in an amount up to 30% by weight.

In a preferred embodiment of the invention, the preparations according to the invention have a fraction of volatile organic components of at most 80% by weight, preferably at most 55% by weight and in particular at most 35% by weight. A preferred subject-matter are thus cosmetic, preferably hair cosmetic, preparations which correspond to the low-VOC standard, i.e. VOC-80 or VOC-55 standard.

Preference is given to the use of the copolymers according to the invention in particular in hairspray preparations which comprise the following constituents:
partially or completely neutralized copolymer according to the invention;
water;
cosmetically customary organic solvent such as, for example, ethanol, isopropanol and dimethoxymethane, in addition also acetone, n-propanol, n-butanol, 2-methoxypropan-1-ol, n-pentane, n-hexane, cyclohexane, n-heptane, n-octane or dichloromethane or mixtures thereof;
cosmetically customary propellant such as, for example, n-propane, isopropane, n-butane, isobutane, 2,2-dimethylbutane, n-pentane, isopentane, dimethyl ether, difluoroethane, fluorotrichloromethane, dichlorodifluoromethane or dichlorotetrafluoroethane, HFC-152 A (1,1-difluoroethane), HFC-134a (1,1,2,2-tetrafluoroethane), N₂, N₂O and CO or mixtures thereof.

To neutralize the copolymers according to the invention and to adjust the pH of the cosmetic, preferably hair cosmetic, preparations, alkanolamines are advantageously used. Examples (INCI) are aminomethylpropanol, diethanolamine, diisopropanolamine, ethanolamine, methylethanolamine, N-lauryldiethanolamine, triethanolamine and triisopropanolamine. It is possible to use alkanolamines carrying either primary amino groups or secondary amino groups.

Furthermore, alkali metal hydroxides (e.g. NaOH, preferably KOH) and other bases can be used for the neutralization (e.g. histidine, arginine, lysine or ethylenediamines, diethylenetriamine, melamine, benzoguanamine). All of the bases given can be used on their own or as a mixture with other bases for the neutralization of acid-containing cosmetic products.

In a preferred embodiment of the invention, amines containing hydroxy groups for the neutralization are chosen from the group consisting of N,N-dimethylethanolamine, N-methyldiethanolamine, triethanolamine, 2-amino-2-methyl-propanol and mixtures thereof.

In this connection, alkanolamines carrying secondary or tertiary amino groups can display advantageous effects.

Accordingly, the present invention provides aqueous cosmetic, preferably skin and/or hair cosmetic, preparations which, besides the at least one copolymer according to the invention and the carrier B, also comprise at least one active ingredient or additive chosen from the group consisting of viscosity-modifying substances, haircare substances, hair-setting substances, silicone compounds, photoprotective substances, fats, oils, waxes, preservatives, pigments, soluble dyes, particulate substances, and surfactants.

In a preferred embodiment, hair cosmetic formulations according to the invention comprise

i) 0.05 to 20% by weight of at least one copolymer according to the invention,
ii) 20 to 99.95% by weight of water and/or alcohol,
iii) 0 to 50% by weight of at least one propellant,
iv) 0 to 5% by weight of at least one emulsifier,
v) 0 to 3% by weight of at least one thickener, and
vi) up to 25% by weight of further constituents.

Alcohol is understood as meaning all of the abovementioned alcohols customary in cosmetics, e.g. ethanol, isopropanol, n-propanol.

### Propellants (Propellant Gases)

Of the specified compounds, the propellants (propellant gases) used are primarily the hydrocarbons, in particular propane, n-butane, n-pentane and mixtures thereof, and also dimethyl ether and difluoroethane. If appropriate, one or more of the specified chlorinated hydrocarbons are co-used in propellant mixtures, but only in small amounts, for example up to 20% by weight, based on the propellant mixture.

The cosmetic, preferably hair cosmetic, preparations according to the invention are also particularly suitable for pump spray preparations without the addition of propellants or else for aerosol sprays with customary compressed gases, such as nitrogen, compressed air or carbon dioxide as propellant.

A hydrous standard aerosol spray formulation comprises, for example, the following constituents:
a copolymer according to the invention neutralized to 100%
a alcohol
water
dimethyl ether and/or propane/n-butane and/or propane/isobutane.

Here, the total amount of volatile organic components is preferably at most 80% by weight, particularly preferably at most 55% by weight, of the preparation.

Preferably, the cosmetic, preferably hair cosmetic, preparations according to the invention comprise at least one copolymer according to the invention, at least one cosmetically acceptable carrier B as defined above and at least one further active ingredient or additive different therefrom which is chosen from cosmetically active ingredients, emulsifiers, surfactants, preservatives, perfume oils, thickeners, hair polymers, hair conditioners, graft polymers, water-soluble or dispersible silicone-containing polymers, photoprotective agents, bleaches, gel formers, care agents, colorants, tints, tanning agents, dyes, pigments, consistency regulators, humectants, refatting agents, collagen, protein hydrolysates, lipids, antioxidants, antifoams, antistats, emollients, lanolin components, protein hydrolysates and softeners.

### Further Polymers

To adjust the properties of cosmetic, preferably hair cosmetic, preparations in a targeted manner, it may be advantageous to use the copolymers according to the invention in mixture with further (hair) cosmetically customary polymers.

In a further preferred embodiment, the composition according to the invention comprises 0.01 to 15% by weight, preferably 0.5 to 10% by weight, of at least one further synthetic or natural nonionic, preferably a film-forming polymer. Natural polymers are also understood as meaning chemically modified polymers of natural origin. Film-forming polymers are understood as meaning those polymers which, when applied in 0.01 to 5% strength aqueous, alcoholic or aqueous-alcoholic solution, are able to deposit a polymer film on the hair.

Suitable such further customary polymers for this are, for example, anionic, cationic, amphoteric, zwitterionic and neutral polymers.

Examples of such further polymers are
copolymers of ethyl acrylate and methacrylic acid, copolymers of N-tert-butylacrylamide, ethyl acrylate and acrylic acid,
polyvinylpyrrolidones,
polyvinylcaprolactams,
polyurethanes,
copolymers of acrylic acid, methyl methacrylate, octylacrylamide, butylaminoethyl methylacrylate and hydroxypropyl methacrylate,
copolymers of vinyl acetate and crotonic acid and/or (vinyl)neodecanoate,
copolymers of vinyl acetate and/or vinyl propionate and N-vinylpyrrolidone,
carboxy-functional copolymers of vinylpyrrolidone, t-butyl acrylate, methacrylic acid,
copolymers of tert-butyl acrylate, methacrylic acid and dimethicone copolyol.

Surprisingly, it has been found that cosmetic, and preferably hair cosmetic, preparations which comprise the polymers A in combination with further polymers have unexpected properties. The cosmetic, and preferably hair cosmetic, preparations according to the invention are superior to the preparations from the prior art especially with regard to the totality of their cosmetic properties.

Copolymers of ethyl acrylate and methacrylic acid (INCI name: Acrylates Copolymer) are available, for example, as commercial products Luviflex®Soft (BASF).

Copolymers of N-tert-butylacrylamide, ethyl acrylate and acrylic acid (INCI name: Acrylates/Acrylamide Copolymer) are available, for example, as commercial products Ultrahold Strong®, Ultrahold (BASF).

Polyvinylpyrrolidones (INCI name: PVP) are available, for example, under the trade names Luviskol®K, Luviskol®K30 (BASF) and PVP K® (ISP).

Polyvinylcaprolactams (INCI: Polyvinylcaprolactams) are available, for example, under the trade name Luviskol Plus® (BASF).

Polyurethanes (INCI: Polyurethane-1) are available, for example, under the trade name Luviset®PUR.

Copolymers of acrylic acid, methyl methacrylate, octylacrylamide, butylaminoethyl methylacrylate, hydroxypropyl methacrylate (INCI: Octylacrylamide/Acrylates/Butyl-aminoethyl Methacrylate Copolymer) are known, for example, under the trade names Amphomer®28-4910 and Amphomer®LV-71 (National Starch).

Copolymers of vinyl acetate and crotonic acid (INCI: VA/Crotonate/Copolymer) are available, for example, under the trade names Luviset®CA 66 (BASF), Resyn®28-1310 (National Starch), Gafset® (GAF) or Aristoflex®A (Celanese).

Copolymers of vinyl acetate, crotonic acid and (vinyl)neodecanoate (INCI: VA/Crotonates/Neodecanoate Copolymer) are available, for example, under the trade names Resyn®28-2930 (National Starch) and Luviset®CAN (BASF).

Copolymers of vinyl acetate and N-vinylpyrrolidone (INCI: PVP/VA) are available, for example, under the trade names Luviskol VA® (BASF) and PVP/VA (ISP).

Carboxyfunctional copolymers of vinylpyrrolidone, t-butyl acrylate, methacrylic acid are available, for example, under the trade name Luviskol®VBM (BASF).

Copolymers of tert-butyl acrylate, methacrylic acid and dimethicone copolyol are available, for example, under the trade name Luviflex®Silk (BASF).

Suitable anionic polymers are homopolymers and copolymers of acrylic acid and methacrylic acid or salts thereof which are different from the polymers A, copolymers of acrylic acid and acrylamide and salts thereof, sodium salts of polyhydroxycarboxylic acids, copolymers of acrylic acid and methacrylic acid with, for example, hydrophobic monomers, e.g. C₄-C₃₀-alkyl esters of (meth)acrylic acid, C₄-C₃₀-alkylvinyl esters, C₄-C₃₀-alkyl vinyl ethers and hyaluronic acid and also further polymers known under the trade names Amerhold®DR-25, Ultrahold®, Luviset®P.U.R., Acronal®, Acudyne®, Lovocryl®, Versatyl®, Amphomer® (28-4910, LV-71), Placise®L53, Gantrez®ES 425, Advantage Plus®, Omnirez®2000, Resyn®28-1310, Resyn®28-2930, Balance®(0/55), Acudyne®255, Aristoflex®A or Eastman AQ®.

In addition, the group of suitable polymers comprises, for example, Balance®CR (National Starch), Balance®47 (National Starch; octylacrylamide/-acrylates/butylaminoethyl methacrylates copolymer), Aquaflex®FX 64 (ISP; isobutylene/ethylmaleimide/hydroxyethylmaleimide copolymer), Aquaflex®SF-40 (ISP/National Starch; VP/vinyl caprolactam/DMAPA acrylates copolymer), Allianz®LT-120 (ISP/Rohm & Haas; acrylate/C1-2 succinate/hydroxyacrylate copolymer), Aquarez® HS (Eastman; Polyester-1).

Also suitable are the polymers under the trade names Diaformer®Z-400 (Clariant; methacryloylethylbetaine/methacrylate copolymer), Diaformer®Z-711 (Clariant; methacryloylethyl N-oxide/methacrylate copolymer), Diaformer®Z-712 (Clariant; methacryloylethyl N-oxide/methacrylate copolymer), Omnirez®2000 (ISP; monoethyl ester of poly(methyl vinyl ether/maleic acid in ethanol), Amphomer®HC (National Starch; acrylate/octylacrylamide copolymer), Amphomer®28-4910 (National Starch; octylacrylamide/acrylate/butylaminoethyl methcrylate copolymer), Advantage®HC 37 (ISP; terpolymer of vinylcaprolactam/vinylpyrrolidone/dimethylaminoethyl methacrylate), Advantage®LC55 and LC80 or LC A and LC E, Advantage®Plus (ISP; VA/butyl maleate/isobornyl acrylate copolymer), Aculyne®258 (Rohm & Haas; acrylate/hydroxy ester acrylate copolymer), Luviset®P.U.R. (BASF, Polyurethane-1), Eastman®AQ 48 (Eastman), Styleze®CC-10 (ISP; VP/DMAPA acrylates copolymer), Styleze® 2000 (ISP; VP/acrylates/Iaurylmethacrylate copolymer), DynamX® (National Starch; polyurethane-14 AMP acrylates copolymer), Resyn®XP (National Starch; acrylates/octylacrylamide copolymer), Fixomer® A-30 (Ondeo Nalco; polymethacrylic acid (and) acrylamidomethylpropanesulfonic acid), Fixate® G-100 (Noveon; AMP acrylates/allyl methacrylate copolymer).

Suitable polymers are also copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® (Rohm +Haas). Particularly suitable polymers are also copolymers of t-butyl acrylate, ethyl acrylate, methacrylic acid (e.g. Luvimer®100P, Luvimer®Pro55) and copolymers of ethyl acrylate and methacrylic acid (e.g. Luvimer®MAE).

Also suitable are crosslinked polymers of acrylic acid, as are available under the INCI name Carbomer. Such crosslinked homopolymers of acrylic acid are commercially available, for example, as Carbopol® (Noveon). Preference is also given to hydrophobically modified crosslinked polyacrylate polymers, such as Carbopol®Ultrez 21 (Noveon). Such further polymers can also be used for modifying the rheology of the preparations, i.e. as thickeners.

Further suitable additional polymers are water-soluble or water-dispersible polyesters, polyureas, polyurethanes, polyurethaneureas, maleic anhydride copolymers reacted, if appropriate, with alcohols, or anionic polysiloxanes.

In addition, polymers suitable for use together with the polymers A are, for example, also cationic and cationogenic polymers. These include, for example,
copolymers of N-vinylpyrrolidone/N-vinylimidazolium salts (available, for example, under the trade names Luviquat®FC, Luviquat®HM, Luviquat®MS, Luviquat®Care, Luviquat® UltraCare (BASF),
copolymers of N-vinylcaprolactam/N-vinylpyrrolidone/N-vinylimidazolium salts (available, for example, under the trade name Luviquat®Hold),
copolymers of N-vinylpyrrolidone/dimethylaminoethyl methacrylate, quaternized with diethyl sulfate (available, for example, under the trade name Luviquat®PQ11),
copolymers of vinylpyrrolidone, methacrylamide, vinylimidazole (Luviset Clear)
cationic cellulose derivatives (polyquaternium-4 and -10),
acrylamide copolymers (polyquaternium-7),
guar hydroxypropyltrimethylammonium chloride (INCI: Hydroxypropyl Guar Hydroxypropyltrimonium Chloride),
polyethyleneimines and salts thereof,
polyvinylamines and salts thereof,
polymers based an dimethyldiallylammonium chloride (Merquat®),
polymers which are formed by reacting polyvinylpyrrolidone with quaternary ammonium compounds (Gafquat®),
hydroxyethylcellulose with cationic groups (Polymer® JR) and
cationic plant-based polymers, e.g. guar polymers, such as the Jaguar® grades from Rhodia.

Suitable as further hair cosmetic polymers are also neutral polymers, such as
polyvinylpyrrolidones,
copolymers of N-vinylpyrrolidone and vinyl acetate and/or vinyl propionate,
polysiloxanes,
polyvinylcaprolactams and
copolymers with N-vinylpyrrolidone,
cellulose derivatives,
polyaspartic acid salts and derivatives,
polyamides, e.g. based on itaconic acid and aliphatic diamines, as described in DE-A-43 33 238.

The abovementioned types of polymer include those known under the trade names Luviskol® (K, VA, Plus), PVP K, PVPNA, Advantage®HC, Luviflex®Swing, H₂OLD®EP-1.

Furthermore, suitable further polymers are also biopolymers, i.e. polymers which are obtained from naturally renewable raw materials and are constructed from natural monomer building blocks, e.g. cellulose derivatives, chitin, chitosan, DNA, hyaluronic acid and RNA derivatives.

Suitable mixing partners for the polymers according to the invention are also zwitterionic polymers, as are disclosed, for example, in the German patent applications DE 39 29 973, DE 21 50 557, DE 28 17 369 and DE 37 08 451, and methacroylethyl-betaine/methacrylate copolymers which are commercially available under the name Amersette® (Amerchol), or copolymers of hydroxyethyl methacrylate, methyl methacrylate, N,N-dimethylaminoethyl methacrylate and acrylic acid (Jordapon®).

Further suitable polymers are also betainic polymers, such as Yukaformers (R205, SM) and Diaformers.

Polymers suitable as mixing partners are also nonionic, siloxane-containing, water-soluble or -dispersible polymers, e.g, polyether siloxanes, such as Tegopren® (Goldschmidt) or Belsil® (Wacker).

Cosmetically and/or Dermatologically Active Ingredients

Suitable cosmetically and/or dermatologically active ingredients are, for example, coloring active ingredients, skin and hair pigmentation agents, tints, tanning agents, bleaches, keratin-hardening substances, antimicrobial active ingredients, photo filter active ingredients, repellant active ingredients, hyperemic substances, keratolytic and keratoplastic substances, antidandruff active ingredients, antiphlogistics, keratinizing substances, antioxidative active ingredients or active ingredients which act as free-radical scavengers, substances which moisten the skin or keep the skin moist, refatting active ingredients, antierythimatous or antiallergic active ingredients and mixtures thereof.

Preferred cosmetic care and active ingredients are AHA acids, fruit acids, ceramides, phytantriol, collagen, vitamins and provitamins, for example vitamin A, E and C, retinol, bisabolol and panthenol. A particularly preferred cosmetic care substance in the preparations according to the invention is panthenol, which is commercially available, for example, as D-Panthenol®USP, D-Panthenol®50 P, D-Panthenol®75 W, D,L-Panthenol®50 W.

Artificially skin-tanning active ingredients which are suitable for tanning the skin without natural or artificial irradiation with UV rays are, for example, dihydroxyacetone, alloxan and walnut shell extract.

Suitable keratin-hardening substances are generally active ingredients as are also used in antiperspirants, such as, for example, potassium aluminum sulfate, aluminum hydroxychloride, aluminum lactate, etc.

Antimicrobial active ingredients are used to destroy microorganisms or to inhibit their growth and thus serve both as preservatives and also as deodorizing substance which reduces the formation or the intensity of body odor. These include, for example, customary preservatives known to the person skilled in the art, such as p-hydroxy-benzoic esters, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine, etc. The preparations according to the invention comprise preferably 0.01 to 5% by weight, particularly preferably 0.05 to 1% by weight, of at least one preservative. Suitable further preservatives are the substances listed in the International Cosmetic Ingredient Dictionary and Handbook, 9th Edition with the function “Preservatives”, e.g. phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin.

### UV Filter Substances

In one embodiment, the preparations according to the invention can comprise oil-soluble and/or water-soluble UVA and/or UVB filters.

The total amount of the filter substances is preferably 0.01 to 10% by weight or from 0.1 to 5% by weight, particularly preferably from 0.2 to 2% by weight, based on the total weight of the preparations.

The majority of the photoprotective agents in the preparations serving to protect the human epidermis consists of compounds which absorb UV light in the UV-B region. For example, the fraction of UV-A absorbers to be used according to the invention is 10 to 90% by weight, preferably 20 to 50% by weight, based on the total amount of UV-B and UV-A absorbing substances.

The UVB filters may be oil-soluble or water-soluble. Advantageous UVB filter substances are, for example:
i) benzimidazolesulfonic acid derivatives, such as, for example, 2-phenylbenzimidazole-5-sulfonic acid and salts thereof;
ii) benzotriazole derivatives, such as, for example, 2,2′-methylenebis(6-(2H-benzotriazol-2-yl)-4-(1,1,3,3-tetramethylbutyl)phenol);
iii) 4-aminobenzoic acid derivatives, preferably 2-ethylhexyl 4-(dimethyl-amino)benzoate, amyl 4-(dimethylamino)benzoate;
iv) esters of benzalmalonic acid, preferably di(2-ethylhexyl) 4-methoxybenzalmalonate;
v) esters of cinnamic acid, preferably 2-ethylhexyl 4-methoxycinnamate, isopentyl 4-methoxycinnamate;
vi) derivatives of benzophenone, preferably 2-hydroxy-4-methoxybenzophenone, 2-hydroxy-4-methoxy-4′-methylbenzophenone, 2,2′-dihydroxy-4-methoxybenzo-phenone;
vii) methylidenecamphor derivatives, preferably 4-methylbenzylidenecamphor, benzylidenecamphor;
viii) triazine derivatives, preferably tris(2-ethylhexyl) 4,4′,4″-(1,3,5-triazine-2,4,6-triylimino)trisbenzoate [INCI: Diethylhexyl Butamido Triazine, UVA-Sorb® HEB (Sigma 3V)] and 2,4,6-tris[anilino(p-carbo-2′-ethyl-t-hexyloxy)]-1,3,5-triazine [INCI: Octyl Triazone, Uvinul® 150 (BASF)].

Water-soluble UVB filter substances to be used advantageously are, for example, sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and salts thereof.

UVA filters to be used advantageously are, for example;
1,4-phenylenedimethinecamphorsulfonic acid derivatives, such as, for example, 3,3′-(1,4-phenylenedimethine)bis(7,7-dimethyl-2-oxobicyclo[2.2.1]heptane-1-methanesulfonic acid and its salts
1,3,5-triazine derivatives, such as 2,4-bis{[(2-ethylhexyloxy)-2-hydroxy]phenyl}-6-(4-methoxyphenyl)-1,3,5-triazine (e.g. Tinosorb®S (Ciba))
dibenzoylmethane derivatives, preferably 4-isopropyldibenzoylmethane, 4-(tert-butyl)-4′-methoxydibenzoylmethane
benzoxazole derivatives, for example 2,4-bis[4-[5-(1,1-dimethylpropyl)benzoxazol-2-yl]phenylimino]-6-[(2-ethylexyl)imino]-1,3,5-triazine (CAS No. 288254-1 6-0, Uvasorb®K2A (3V Sigma))
hydroxybenzophenones, for example hexyl 2-(4′-diethylamino-2′-hydroxy-benzoyl)benzoate (also: aminobenzophenone) (Uvinul®A Plus (BASF))

In addition, it may, if appropriate, be advantageous according to the invention to provide preparations with further UVA and/or UVB filters, for example certain salicylic acid derivatives, such as 4-isopropylbenzyl salicylate, 2-ethylhexyl salicylate, octyl salicylate, homomethyl salicylate. The total amount of salicylic acid derivatives in the cosmetic preparations is advantageously chosen from the range from 0.1-15.0% by weight, preferably 0.3-10.0% by weight, based on the total weight of the preparations. A further photoprotective filter to be used advantageously according to the invention is ethylhexyl 2-cyano-3,3-diphenylacrylate (octocrylene, Uvinul®N 539 (BASF)).

The table below lists by way of example some of the photoprotective filters suitable for use in the preparations according to the invention:

| No. | Substance | CAS No. |
|---|---|---|
| 1 | 4-Aminobenzoic acid | 150-13-0 |
| 2 | 3-(4′-Trimethylammonium)benzylidenebornan-2-one methyl | 52793-97-2 |
| | sulfate | |
| 3 | 3,3,5-Trimethylcyclohexyl salicylate (homosalate) | 118-56-9 |
| 4 | 2-Hydroxy-4-methoxybenzophenone (oxybenzone) | 131-57-7 |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl-2- | 90457-82-2 |
| | oxobicyclo[2,2.1]heptane-1-methanesulfonic acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methylbenzophenone (mexenone) | 1641-17-4 |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3,- | 103597-45-1 |
| | tetramethylbutyl)phenol] | |
| 30 | 2,2′-(1,4-Phenylene)bis-1H-benzimidazole-4,6-disulfonic acid, | 180898-37-7 |
| | Na salt | |
| 31 | 2,4-bis[4-(2-Ethylhexyloxy)-2-hydroxy]phenyl- | 187393-00-6 |
| | 6-(4-methoxyphenyl)(1,3,5)triazine | |
| 32 | 3-(4-Methylbenzylidene)camphor | 36861-47-9 |
| 33 | Polyethoxyethyl4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone 5,5′- | 3121-60-6 |
| | disodium sulfonate | |
| 36 | Benzoic acid, 2-[4-(diethylamino)-2-hydroxybenzoyl]-, hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

Suitable UV photoprotective filters with the CAS No. 113010-52-9 are commercially available, for example, under the name Uvinul ®P 25,

Polymeric or polymer-bound filter substances can also be used according to the invention.

Metal oxides such as titanium dioxide or zinc oxide can likewise be used advantageously for protecting against harmful solar radiation. Their effect is essentially based on reflection, scattering and absorption of the harmful UV radiation and essentially depends on the primary particle size of the metal oxides. The cosmetic preparations according to the invention can, furthermore, advantageously comprise inorganic pigments based on metal oxides and/or other metal compounds which are insoluble or sparingly soluble in water, chosen from the group of oxides of zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. They are particularly preferably pigments based on ZnO.

The inorganic pigments can here be present in coated form, i.e. that they are treated superficially. This surface treatment can, for example, consist in providing the pigments with a thin hydrophobic layer by a method known per se, as described in DE-A-33 14 742.

Photoprotective agents suitable for use in the preparations according to the invention are the compounds specified in EP-A 1 084 696 in paragraphs [0036] to [0053], which is hereby incorporated in its entirety at this point by reference. Of suitability for the use according to the invention are all UV photoprotective filters which are specified in Annex 7 (to §3b) of the German Cosmetics Directive under “Ultraviolet filters for cosmetic compositions”.

The list of specified UV photoprotective filters which can be used in the preparations according to the invention is not exhaustive.

### Thickeners

Suitable thickeners are specified in “Kosmetik und Hygiene von Kopf bis Fuβ” [Cosmetics and hygiene from head to foot], Ed. W. Umbach, 3rd Edition, Wiley-VCH, 2004, pp. 235-236, which is hereby incorporated in its entirety at this point by reference.

Consistency regulators allow the desired viscosity of, for example, shampoos to be set. Thickeners which have a viscosity-increasing effect due to the surfactant micelles increasing in size or due to swelling of the water phase originate from chemically very different classes of substances.

Suitable thickeners for the preparations according to the invention are, for example, crosslinked polyacrylic acids and derivatives thereof, polysaccharides such as xanthan gum, guar guar, agar agar, alginates or tyloses, cellulose derivatives, e.g. carboxymethylcellulose or hydroxycarboxymethylcellulose, also higher molecular weight polyethylene glycol mono- and diesters of fatty acids, fatty alcohols, monoglycerides and fatty acids, polyvinyl alcohol and polyvinylpyrrolidone.

Suitable thickeners are commercially available under the trade names Carbopol® (Noveon), Ultrez® (Noveon), Luvigel® EM (BASF), Capigel®98 (Seppic), Synthalene® (Sigma), the Aculyn® grades from Rohm and Haas, such as Aculyn® 22 (copolymer of acrylates and methacrylic acid ethoxylates with stearyl radical (20 EO units)) and Aculyn® 28 (copolymer of acrylates and methacrylic acid ethoxylates with behenyl radical (25 EO units)).

Suitable thickeners are also, for example, Aerosil grades (hydrophilic silicas), polyacrylamides, polyvinyl alcohol and polyvinylpyrrolidone, surfactants such as, for example, ethoxylated fatty acid glycerides, esters of fatty acids with polyols, such as, for example, pentaerythritol or trimethylolpropane, fatty alcohol ethoxylates with a narrowed homolog distribution or alkyl oligoglucosides, and electrolytes, such as sodium chloride and ammonium chloride.

Particularly preferred thickeners for producing gels are Ultrez®21, Aculyn®28, Luvigel® EM and Capigel®98.

Particularly in the case of more highly concentrated shampoo formulations it is also possible, to regulate the consistency, to add substances which reduce the viscosity of the formulation, such as, for example, propylene glycol and glycerol. These substances influence the product properties only slightly.

### Gel Formers

If the use of gel formers is desired for the preparations according to the invention, then all gel formers customary in cosmetics can be used. These include slightly crosslinked polyacrylic acid, for example Carbomer (INCI), cellulose derivatives, e.g. Hydroxypropylcellulose, Hydroxyethylcellulose, cationically modified celluloses, polysaccharides, e.g. Xanthan gum, Caprylic/Capric Triglyceride, Sodium Acrylates Copolymer, Polyquaternium-32 (and) Paraffinum Liquidum (INCI), Sodium Acrylates Copolymer (and) Paraffinum Liquidum (and) PPG-1 Trideceth-6, Acrylamidopropyl Trimonium Chloride/Acrylamide Copolymer, Steareth-10 Allyl Ether Acrylates Copolymer, Polyquaternium-37 (and) Paraffinum Liquidum (and) PPG-1 Trideceth-6, Polyquaternium 37 (and) Propylene Glycole Dicaprate Dicaprylate (and) PPG-1 Trideceth-6, Polyquaternium-7, Polyquaternium-44.

### Emulsifiers

Suitable emulsifiers are, for example, nonionogenic surfactants from at least one of the following groups:
i) Addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
ii) C12/18 fatty acid mono- and diesters of addition products of from 1 to 30 mol of ethylene oxide onto glycerol;
iii) glycerol mono- and diesters and sorbitan mono- and diesters of saturated and unsaturated fatty acids having 6 to 22 carbon atoms and ethylene oxide addition products thereof;
iv) alkyl mono- and oligoglycosides having 8 to 22 carbon atoms in the alkyl radical and ethoxylated analogs thereof;
v) addition products of from 15 to 60 mol of ethylene oxide onto castor oil and/or hydrogenated castor oil;
vi) polyol and in particular polyglycerol esters, such as, for example, polyglycerol polyricinoleate, polyglycerol poly-12-hydroxystearate or polyglycerol dimerate. Likewise suitable are mixtures of compounds from two or more of these classes of substance;
vii) addition products of from 2 to 15 mol of ethylene oxide onto castor oil and/or hydrogenated castor oil;
viii) partial esters based on linear, branched, unsaturated or saturated C₆/₂₂-fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose);
ix) mono-, di- and trialkyl phosphates, and mono-, di- and/or tri-PEG alkyl phosphates and salts thereof;
x) wool wax alcohols;
xi) polysiloxane-polyalkyl-polyether copolymers and corresponding derivatives;
xii) mixed esters of pentaerythritol, fatty acids, citric acid and fatty alcohol according to German patent 1165574 and/or mixed esters of fatty acids having 6 to 22 carbon atoms, methylglycose and polyols, preferably glycerol or polyglycerol and
xiii) polyalkylene glycols.

The addition products of ethylene oxide and/or of propylene oxide onto fatty alcohols, fatty acids, alkylphenols, glycerol mono- and diesters, and also sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C₁₂ to C₁₈-fatty acid mono- and diesters of addition products of ethylene oxide onto glycerol are known from German patent 2024051 as refatting agents for cosmetic preparations. C₈ to C₁₈-alkyl mono- and oligoglycosides, their preparation and their use are known from the prior art. Their preparation takes place in particular by reacting glucose or oligosaccharides with primary alcohols having 8 to 18 carbon atoms. With regard to the glycoside ester, both monoglycosides in which a cyclic sugar radical is glycosidically bonded to the fatty alcohol, and also oligomeric glycosides with a degree of oligomerization up to preferably about 8 are suitable. The degree of oligomerization here is a statistical average value which is based on a homolog distribution customary for such technical-grade products.

In addition, zwitterionic surfactants can be used as emulsifiers. Zwitterionic surfactants is the term used to refer to those surface-active compounds which carry at least one quaternary ammonium group and at least one carboxylate group and/or one sulfonate group in the molecule. Particularly suitable zwitterionic surfactants are the so-called betaines, such as the N-alkyl-N,N-dimethylammonium glycinates, for example cocoa alkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines each having 8 to 18 carbon atoms in the alkyl or acyl group, and cocoacylaminoethyl hydroxyethylcarboxymethyl glycinate.

Particular preference is given to the fatty acid amide derivative known under the CTFA name Cocamidopropyl Betaine. Likewise suitable emulsifiers are ampholytic surfactants. Ampholytic surfactants are understood as meaning those surface-active compounds which, apart from a C₈ to C₁₈-alkyl or -acyl group in the molecule, comprise at least one free amino group and at least one —COOH and/or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂ to C₁₈-acylsamosine.

Besides the ampholytic emulsifiers, quaternary emulsifiers are also suitable, particular preference being given to those of the esterquat type, preferably methyl-quaternized difatty acid triethanolamine ester salts.

### Antioxidants

An additional content of antioxidants in the preparations may be advantageous. According to the invention, antioxidants which may be used are all antioxidants which are customary or suitable for cosmetic applications. The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, γ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. β-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the abovementioned antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to provide these in concentrations of from 0.001 to 10% by weight, based on the total weight of the preparation.

If vitamin A or vitamin A derivatives, or carotenes or derivatives thereof are the antioxidant or the antioxidants, it is advantageous to provide these in concentrations of from 0.001 to 10% by weight, based on the total weight of the preparation.

### Perfume Oils

The cosmetic, preferably hair cosmetic, preparations can comprise perfume oils. Perfume oils which may be mentioned are, for example, mixtures of natural and synthetic fragrances. Natural fragrances are extracts of flowers (lily, lavender, rose, jasmine, neroli, ylang-ylang), stems and leaves (geranium, patchouli, petitgrain), fruits (anise, coriander, caraway, juniper), fruit peels (bergamot, lemon, orange), roots (mace, angelica, celery, cardamom, costus, iris, calmus), woods (pine wood, sandalwood, guaiac wood, cedar wood, rosewood), herbs and grasses (tarragon, lemongrass, sage, thyme), needles and branches (spruce, fir, pine, dwarf pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type, Fragrance compounds of the ester type are, for example, benzyl acetate, phenoxyethyl isobutyrate, 4-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonat, the ketones include, for example, the ionones, α-isomethylionone and methyl cedryl ketone, the alcohols include anethol, citronellol, eugenol, isoeugenol, geraniol, linalool, phenylethyl alcohol and terioneol, and the hydrocarbons include primarily the terpenes and balsams. However, preference is given to using mixtures of different fragrances which together produce a pleasing scent note, Essential oils of lower volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, vertofix coeur, iso-E-super, Fixolide NP, evernyl, iraldein gamma, phenylacetic acid, geranyl acetate, benzyl acetate, rose oxide, romillat, irotyl and floramat alone or in mixtures.

### Superfatting Agents

Superfatting agents which may be used are substances such as, for example, lanolin and lecithin, and polyethoxylated or acylated lanolin and lecithin derivatives, polyol fatty acid esters, monoglycerides and fatty acid alkanolamides, the latter serving as foam stabilizers at the same time.

### Silicone Compounds

In one embodiment, the preparations according to the invention comprise, as haircare additive, at least one silicone compound in an amount of preferably 0.01 to 15% by weight, particularly preferably from 0.1 to 5% by weight. The silicone compounds comprise volatile and nonvolatile silicones and silicones which are insoluble or soluble in the composition. In one embodiment, they are high molecular weight silicones with a viscosity of from 1000 to 2 000 000 cSt at 25° C., preferably 10 000 to 1 800 000 or 100 000 to 1 500 000. The silicone compounds comprise polyalkyl- and polyaryl-siloxanes, in particular with methyl, ethyl, propyl, phenyl, methylphenyl and phenyl-methyl groups. Preference is given to polydimethylsiloxanes, polydiethylsiloxanes, polymethylphenylsilexanes. Preference is also given to shine-imparting, arylated silicones with a refractive index of at least 1.46, or at least 1.52. The silicone compounds comprise in particular the substances with the INCI names cyclomethicone, dimethicone, dimethiconol, dimethicone copolyol, phenyl trimethicone, amodimethicone, trimethylsilylamodimethicone, stearyl siloxysilicate, polymethyl-silsesquioxane, dimethicone crosspolymer. Also suitable are silicone resins and silicone elastomers, which are highly crosslinked siloxanes.

Preferred silicones are cyclic dimethylsiloxanes, linear polydimethylsiloxanes, block polymers of polydimethylsiloxane and polyethylene oxide and/or polypropylene oxide, polydimethylsiloxanes with terminal or lateral polyethylene oxide or polypropylene oxide radicals, polydimethylsiloxanes with terminal hydroxyl groups, phenyl-substituted polydimethylsiloxanes, silicone emulsions, silicone elastomers, silicone waxes, silicone gums and amino-substituted silicones (CTFA: amodimethicones).

### Hair Conditioners

In one embodiment, the preparations according to the invention comprise 0.01 to 20% by weight, preferably from 0.05 to 10% by weight, particularly preferably from 0.1 to 5% by weight, of at least one conditioner.

Conditioning agents preferred according to the invention are, for example, all compounds which are listed in the International Cosmetic Ingredient Dictionary and Handbook (Volume 4, Editor: R. C. Pepe, J. A. Wenninger, G. N. McEwen, The Cosmetic, Toiletry, and Fragrance Association, 9th Edition, 2002) under Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all of the compounds listed in EP-A 934 956 (pp, 11-13) under “water soluble conditioning agent” and “oil soluble conditioning agent”. Further advantageous conditioning agents are, for example, the compounds referred to in accordance with INCI as polyquaternium (in particular Polyquaternium-1 to Polyquaternium-56). Suitable conditioning agents include, for example, also polymeric quaternary ammonium compounds, cationic cellulose derivatives, chitosan derivatives and polysaccharides.

The conditioner is preferably chosen from betaine, panthenol, panthenyl ethyl ether, sorbitol, protein hydrolysates, plant extracts; A-B block copolymers of alkyl acrylates and alkyl methacrylates; A-B block copolymers of alkylmethacrylates and acrylonitrile; A-B-A block copolymers of lactide and ethylene oxide; A-B-A block copolymers of caprolactone and ethylene oxide; A-B-C block copolymers of alkylene or alkadiene compounds, styrene and alkyl methacrylates; A-B-C block copolymers of acrylic acid, styrene and alkyl methacrylates, star-shaped block copolymers, hyperbranched polymers, dendrimers, intrinsically electrically conductive 3,4-polyethylenedioxythiophenes and intrinsically electrically conductive polyanilines.

Further conditioners advantageous according to the invention are cellulose derivatives and quaternized guar gum derivatives, in particular guar hydroxypropylammonium chloride (e.g. Jaguar Excel®, Jaguar C 162® (Rhodia), CAS 65497-29-2, CAS 39421-75-5).

Nonionic poly-N-vinylpyrrolidone/polyvinyl acetate copolymers (e.g, Luviskol®VA 64 (BASF)), anionic acrylate copolymers (e.g. Luviflex®Soft (BASF)), and/or amphoteric amide/acrylate/methacrylate copolymers (e.g. Amphomer® (National Starch)) can also be used advantageously according to the invention as conditioners.

### Hydrotropes

To improve the flow behavior, hydrotropes, such as, for example, ethanol, isopropyl alcohol, or polyols, can also be used. Polyols which are suitable here have preferably 2 to 15 carbon atoms and at least two hydroxyl groups. Typical examples are
i) glycerol;
ii) alkylene glycols, such as, for example, ethylene glycol, diethylene glycol, propylene glycol, butylene glycol, hexylene glycol, and polyethylene glycols with an average molecular weight of from 100 to 1000 daltons;
iii) technical-grade oligoglycerol mixtures with a degree of self-condensation of from 1.5 to 10, such as, for example, technical-grade diglycerol mixtures with a diglycerol content of from 40 to 50% by weight;
iv) methylol compounds, such as, in particular, trimethylolethane, trimethylolpropane, trimethylolbutane, pentaerythritol and dipentaerythritol;
v) lower alkyl glucosides, in particular those with 1 to 8 carbon atoms in the alkyl radical, such as, for example, methyl and butyl glucoside;
vi) sugar alcohols having 5 to 12 carbon atoms, such as, for example, sorbitol or mannitol;
vii) sugars having 5 to 12 carbon atoms, such as, for example, glucose or sucrose;
viii) amino sugars, such as, for example, glucamine.

### Oils, Fats and Waxes

The cosmetic, preferably hair cosmetic, preparations according to the invention can also comprise oils, fats or waxes. These are advantageously chosen from the group of lecithins and fatty acid triglycerides, namely the triglycerol esters of saturated and/or unsaturated, branched and/or unbranched alkanecarboxylic acids with a chain length of from 8 to 24, in particular 12 to 18, carbon atoms. The fatty acid triglycerides can, for example, be chosen advantageously from the group of synthetic, semisynthetic and natural oils, such as, for example, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like. Further polar oil components can be chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, and from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, dicaprylyl carbonate (Cetiol CC) and cocoglycerides (Myritol 331), butylene glycol dicaprylate/dicaprate and dibutyl adipate, and synthetic, semisynthetic and natural mixtures of such esters, such as, for example, jojoba oil.

In addition, one or more oil components can be chosen advantageously from the group of branched and unbranched hydrocarbons and hydrocarbon waxes, silicone oils, dialkyl ethers, the group of saturated or unsaturated, branched or unbranched alcohols. Any mixtures of such oil and wax components are also to be used advantageously for the purposes of the present invention. It may also, if appropriate, be advantageous to use waxes, for example cetyl palmitate, as the sole lipid component of the oil phase.

According to the invention, the oil component is advantageously chosen from the group consisting of 2-ethylhexyl isostearate, octyldodecanol, isotridecyl isononanoate, isoeicosane, 2-ethylhexyl cocoate, C12-15-alkylbenzoate, caprylic-capric triglyceride, dicaprylyl ether.

Mixtures of C12-15-alkyl benzoate and 2-ethylhexyl isostearate, mixtures of C12-15-alkyl benzoate and isotridecyl isononanoate, and mixtures of C12-C15-alkyl benzoate, 2-ethylhexyl isostearate and isotridecyl isononanoate are advantageous according to the invention.

According to the invention, particular preference is given to using fatty acid triglycerides, in particular soybean oil and/or almond oil, as oils with a polarity of from 5 to 50 mN/m.

In addition, the oil phase can advantageously be chosen from the group of Guerbet alcohols. These are liquid even at low temperatures and cause virtually no skin irritations. They can be used advantageously as fatting, superfatting and also refatting constituents in cosmetic compositions.

The use of Guerbet alcohols in cosmetics is known per se.

Guerbet alcohols preferred according to the invention are 2-butyloctanol (available commercially, for example, as Isofol®12 (Condea)) and 2-hexyldecanol (available commercially, for example, as Isofol®16 (Condea)). According to the invention, mixtures of Guerbet alcohols according to the invention are also to be used advantageously, such as, for example, mixtures of 2-butyloctanol and 2-hexyldecanol (commercially available, for example, as Isofol®14 (Condea)), Any mixtures of such oil and wax components are also to be used advantageously for the purposes of the present invention. Among the polyolefins, polydecenes are the preferred substances.

Fat and/or wax components to be used advantageously according to the invention can be chosen from the group of vegetable waxes, animal waxes, mineral waxes and petrochemical waxes. For example, candelilla wax, carnauba wax, japan wax, esparto grass wax, cork wax, guaruma wax, ricegerm oil wax, sugarcane wax, berry wax, ouricury wax, montan wax, jojoba wax, shea butter, beeswax, shellac wax, spermaceti, lanolin (wool wax), uropygial grease, ceresine, ozokerite (earth wax), paraffin waxes and microwaxes are advantageous.

Further advantageous fat and/or wax components are chemically modified waxes and synthetic waxes, such as, for example, Syncrowax®HRC (glyceryl tribehenate), and Syncrowax®AW 1 C(C₁₈₋₃₆-fatty acid) and montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates, such as, for example, Tegosoft®CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, O₂₀₋₄₀-alkyl stearate, C₂₀₋₄₀-alkyl hydroxystearoylstearate and/or glycol montanate. Furthermore, certain organosilicon compounds which have similar physical properties to the specified fat and/or wax components, such as, for example, stearoxytrimethylsilane, are also advantageous. According to the invention, the fat and/or wax components can be used either individually or as a mixture in the compositions.

Any mixtures of such oil and wax components are also to be used advantageously for the purposes of the present invention.

The oil phase is advantageously chosen from the group consisting of 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric triglyceride, dicaprylyl ether.

Mixtures of octyldodecanol, caprylic/capric triglyceride, dicaprylyl ether, dicaprylyl carbonate, cocoglycerides or mixtures of C₁₂₋₁₆-alkyl benzoate and 2-ethylhexyl isostearate, mixtures of C₁₂₋₁₅-alkyl benzoate and butylene glycol dicaprylate/dicaprate and mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene are to be used advantageously for the purposes of the present invention.

The oil component is also advantageously chosen from the group of phospholipids. According to the invention, paraffin oil advantageous according to the invention which may be used is Merkur® white oil Pharma 40 from Merkur Vaseline, Shell Ondina® 917, Shell Ondina® 927, Shell Oil 4222, Shell Ondina®933 from Shell & DEA Oil, Pionier® 6301 S, Pionier® 2071 (Hansen & Rosenthal).

Suitable cosmetically compatible oil and fat components are described in Karl-Heinz Schrader, Grundlagen und Rezepturen der Kosmetika [Fundamentals and formulations of cosmetics], 2nd Edition, Verlag Hüthig, Heidelberg, pp. 319-355, which is hereby incorporated in its entirety by reference.

The content of oils, fats and waxes is at most 30% by weight, preferably 20% by weight, further preferably at most 10% by weight, based on the total weight of the composition.

### Pigments

In one embodiment, the preparations according to the invention comprise at least one pigment. These may be colored pigments which impart color effects to the product mass or to the hair, or they may be luster effect pigments which impart luster effects to the product mass or to the hair. The color effects or luster effects on the hair are preferably temporary, i.e. they remain on the hair until the next hair wash and can be removed again by washing the hair with customary shampoos.

The pigments are present in the product mass in undissolved form and may be present in an amount of from 0.01 to 25% by weight, particularly preferably from 5 to 15% by weight. The preferred particle size is 1 to 200 μm, in particular 3 to 150 μm, particularly preferably 10 to 100 μm. The pigments are colorants which are virtually insoluble in the application medium and may be inorganic or organic. Inorganic-organic mixed pigments are also possible. Preference is given to inorganic pigments. The advantage of the inorganic pigments is their excellent stability to light, weather and temperature. The inorganic pigments may be of natural origin, prepared for example from chalk, ochre, umber, green earth, burnt sienna or graphite. The pigments may be white pigments, such as; for example, titanium dioxide or zinc oxide, black pigments, such as, for example, iron oxide black, colored pigments, such as, for example, ultramarine or iron oxide red, luster pigments, metal effect pigments, pearlescent pigments, and fluorescent or phosphorescent pigments, with preferably at least one pigment being a colored, non-white pigment.

Metal oxides, hydroxides and oxide hydrates, mixed phase pigments, sulfur-containing silicates, metal sulfides, complex metal cyanides, metal sulfates, chromates and molybdates, and the metals themselves (bronze pigments) are suitable. Titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI-77007, Pigment Blue 29), chromium oxide hydrate (C177289), iron blue (ferric ferrocyanide, CI77510), carmine (cochineal) are particularly suitable.

Particular preference is given to pearlescent pigments and colored pigments based on mica which are coated with a metal oxide or a metal oxychloride such as titanium dioxide or bismuth oxychloride and, if appropriate, further color-imparting substances, such as iron oxides, iron blue, ultramarine, carmine etc. and where the color can be determined by varying the layer thickness. Such pigments are sold, for example, under the trade names Rona®, Colorona®, Dichrona® and Timiron® by Merck, Germany.

Organic pigments are, for example, the natural pigments sepia, gamboge, bone charcoal, Cassel brown, indigo, chlorophyll and other plant pigments. Synthetic organic pigments are, for example, azopigments, anthraquinoids, indigoids, dioxazine, quinacridone, phthalocyanine, isoindolinone, perylene and perinone, metal complex, alkali blue and diketopyrrolopyrrole pigments.

In one embodiment, the preparations according to the invention comprise 0.01 to 10% by weight, particularly preferably from 0.05 to 5% by weight, of at least one particulate substance. Suitable substances are, for example, substances which are solid at room temperature (25° C.) and are in the form of particles. For example, silica, silicates, aluminates, clay earths, mica, salts, in particular inorganic metal salts, metal oxides, e.g. titanium dioxide, minerals and polymer particles are suitable.

The particles are present in the composition in undissolved, preferably stably dispersed, form and, following application to the application surface and evaporation of the solvent, can be deposited in solid form.

Preferred particulate substances are silica (silica gel, silicon dioxide) and metal salts, in particular inorganic metal salts, with silica being particularly preferred. Metal salts are, for example, alkali metal or alkaline earth metal halides, such as sodium chloride or potassium chloride; alkali metal or alkaline earth metal sulfates, such as sodium sulfate or magnesium sulfate.

Suitable repellent active ingredients are compounds which are able to keep off or drive away certain animals, in particular insects, from people. These include, for example, 2-ethyl-1,3-hexanediol, N,N-diethyl-m-toluamide etc.

Suitable hyperemic substances, which stimulate the circulation of blood through the skin, are, for example, essential oils, such as dwarf pine, lavender, rosemary, juniper berry, horse chestnut extract, birch leaf extract, hay flower extract, ethyl acetate, camphor, menthol, peppermint oil, rosemary extract, eucalyptus oil, etc.

Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc.

Suitable antiphlogistics, which counteract skin irritations, are, for example, allantoin, bisabolol, dragosantol, camomile extract, panthenol, etc.

### Application Form

In a preferred embodiment, the preparations according to the invention are sprayable, for example as aerosol or pump spray preparation.

The preparations according to the invention can be used in various forms, such as, for example, as lotion, as nonaerosol spray lotion, which is used by means of a mechanical device for spraying, as aerosol spray which is sprayed using a propellant, as aerosol foam or as nonaerosol foam, which is present in combination with a suitable mechanical device for foaming the composition, as hair cream, as hair wax, as gel, as liquid gel, as sprayable gel or as foam gel.

Use in the form of a lotion thickened with a customary thickener is also possible.

In one embodiment, the composition according to the invention is in the form of a gel, in the form of a viscous lotion or in the form of a spray gel which is sprayed using a mechanical device, and comprises at least one of the abovementioned thickeners in an amount of from preferably 0.05 to 10% by weight, particularly preferably from 0.1 to 2% by weight and has a viscosity of at least 250 mPas. The viscosity of the gel is preferably from 500 to 50 000 mPas, particularly preferably from 1000 to 15 000 mPas at 25° C.

In another embodiment, the preparation according to the invention is in the form of an O/W emulsion, a W/O emulsion or a microemulsion and comprises at least one of the abovementioned oils or waxes emulsified in water, and at least one cosmetically customary surfactant.

In a preferred embodiment, the preparation according to the invention is in the form of a spray product, either in combination with a mechanical pump spray device or in combination with at least one of the abovementioned propellants. A preferred aerosol spray additionally comprises propellants in an amount such that the total amount of the volatile organic components does not exceed 80% by weight, in particular 55% by weight of the preparation and is bottled in a pressurized container.

A nonaerosol hairspray is sprayed using a suitable mechanically operated spray device. Mechanical spray devices are understood as meaning those devices which permit the spraying of a composition without use of a propellant. A suitable mechanical spray device which may be used is, for example, a spray pump or an elastic container provided with a spray valve in which the cosmetic preparation according to the invention is bottled under pressure, where the elastic container expands and from which the composition is continuously dispensed as a result of the contraction of the elastic container from opening the spray valve.

In a further embodiment, the preparation according to the invention is in the form of a foamable product (mousse) in combination with a devices for foaming, comprises at least one customary foam-imparting substance known for this purpose, e.g. at least one foam-forming surfactant or at least one foam-forming polymer. Devices for foaming are understood as meaning those devices which permit the foaming of a liquid with or without use of a propellant. A suitable mechanical foam device which can be used is, for example, a commercially customary pump foamer or an aerosol foam head. The product is present either in combination with a mechanical pump foam device (pump foam) or in combination with at least one propellant (aerosol foam) in an amount of from preferably 1 to 20% by weight, in particular from 2 to 10% by weight. Propellants are, for example, chosen from propane, butane, dimethyl ether and fluorinated hydrocarbons.

The invention thus provides a cosmetic, preferably hair cosmetic preparation in the form of a spray product, where the preparation is present either in combination with a mechanical pump spray device or in combination with at least one propellant chosen from the group consisting of propane, butane, dimethyl ether, fluorinated hydrocarbons and mixtures thereof.

The composition is foamed directly prior to use and incorporated into the hair as foam and can then be rinsed out or left in the hair without rinsing out.

A formulation preferred according to the invention for aerosol hair foams comprises
i) 0.1 to 10% by weight of at least one copolymer according to the invention,
ii) 55 to 99.8% by weight of water and alcohol,
iii) 5 to 20% by weight of a propellant,
iv) 0.1 to 5% by weight of an emulsifier,
v) 0 to 10% by weight of further constituents,
where the total amount of VOC is at most 80% by weight and preferably at most 55% by weight.

Emulsifiers which can be used are all emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
i) 0.1 to 10% by weight of copolymer according to the invention,
ii) 80 to 99.85% by weight of water and alcohol,
iii) 0 to 3% by weight, preferably 0.05 to 2% by weight, of a gel former,
iv) 0 to 20% by weight of further constituents,
where the total amount of VOC is at most 80% by weight and preferably 55% by weight.

When preparing gels based on the copolymer according to the invention, customary gel formers can be used, for example, in order to establish specific rheological or other applications-related properties. Gel formers which can be used are all gel formers customary in cosmetics. These include slightly crosslinked polyacrylic acid, for example carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) Paraffinum Liquidum (INCI), sodium acrylate copolymers (and) Paraffinum Liquidum (and) PPG-1 trideceth-6, acrylamidopropyltrimonium chloride/acrylamide copolymers, steareth-10 allyl ether acrylate copolymers, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44. Crosslinked homopolymers of acrylic acid suitable as gel formers are commercially available, for example, under the name Carbopol® (Noveon). Preference is also given to hydrophobically modified crosslinked polyacrylate polymers, such as Carbopol®Ultrez 21 (Noveon). Further examples of anionic polymers suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are commercially available as Aculyn® (Rohm and Haas).

In a further embodiment, the preparation according to the invention is in the form of a hair wax, i.e. it has wax-like consistency and comprises at least one of the abovementioned waxes in an amount of from preferably 0.5 to 30% by weight, and if appropriate further water-insoluble substances. The wax-like consistency is preferably characterized in that the needle penetration number (unit of measurement 0.1 mm, test weight 100 g, test time 5 s, test temperature 25° C.; in accordance with DIN 51 579) is greater than or equal to 10, particularly preferably greater than or equal to 20 and that the solidification point of the product is preferably greater than or equal to 30° C. and less than or equal to 70° C., is particularly preferably in the range from 40 to 55° C. Suitable waxes and water-insoluble substances are, in particular, emulsifiers with a HLB value below 7, silicone oils, silicone waxes, waxes (e.g. wax alcohols, wax acids, wax esters, and in particular natural waxes, such as beeswax, carnauba wax etc.), fatty alcohols, fatty acids, fatty acid esters or hydrophilic waxes, such as, for example, high molecular weight polyethylene glycols with a molecular weight of from 800 to 20 000 g/mol, preferably from 2000 to 10 000 g/mol.

If the cosmetic, preferably hair cosmetic, preparation according to the invention is in the form of a hair lotion, then it is present as an essentially non-viscous or low-viscosity, flowable solution, dispersion or emulsion with a content of at least 10% by weight, preferably 20 to 95% by weight, of a cosmetically compatible alcohol. Alcohols which can be used are, in particular, the lower alcohols having 1 to 4 carbon atoms customarily used for cosmetic purposes, e.g. ethanol and isopropanol.

If the hair cosmetic preparation according to the invention is in the form of a hair cream, then it is preferably in the form of an emulsion and comprises either additionally viscosity-imparting ingredients in an amount of from 0.1 to 10% by weight, or the required viscosity and creamy consistency is built up through micelle formation with the help of suitable emulsifiers, fatty acids, fatty alcohols or waxes. in the customary way.

The copolymers according to the invention can be used as conditioners in cosmetic preparations.

The copolymers according to the invention can preferably be used in shampoo formulations as setting and/or conditioning agents. Preferred shampoo formulations comprise
i) 0.05 to 10% by weight of at least one copolymer according to the invention,
ii) 25 to 94.95% by weight of water,
iii) 5 to 50% by weight of surfactants,
iv) 0 to 5% by weight of a further conditioning agent,
v) 0 to 10% by weight of further cosmetic constituents.

In the shampoo formulations, all of the anionic, neutral, amphoteric or cationic surfactants customarily used in shampoos can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate are suitable.

Suitable amophoteric surfactants are, for example, alkylbetaines, alkylaminopropyl-betaines, alkylsulfobetaines, alkyl glycinates, alkyl carboxyglycinates, alkyl amphoacetates or propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioners can be used in combination with the polymers A. These include, for example, the abovementioned cationic polymers with the INCI name Polyquaternium, in particular copolymers of vinylpyrrolidone/N-vinylimidazolium salts (Luviquat®FC, Luviquat®HM, Luviquat®MS, Luviquat®Care, Luviquat®Ultracare), copolymers of N-vinylpyrrolidone/dimethylaminoethyl methacrylate, quaternized with diethyl sulfate (Luviquat®PQ 11), copolymers of N-vinylcaprolactam/N-vinylpyrrolidone/N-vinyl-imidazolium salts (Luviquat®Hold); cationic cellulose derivatives (polyquaternium-4 and -10), acrylamide copolymers (polyquaternium-7). It is also possible to use protein hydrolysates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and aminofunctional silicone compounds, such as amodimethicones (CTFA). In addition, cationic guar derivatives, such as guar hydroxypropyltrimonium chloride (INCI) can be used.

### Measurement Methods

### Determination of the K Value

The K values are measured in accordance with Fikentscher, Cellulosechemie, Vol. 13, pp. 58 to 64 (1932) at 25° C. in ethanol or N-methylpyrrolidone (NMP) solution and are a measure of the molecular weight. The ethanol or NMP solutions of the polymers each comprise 1 g of the particular copolymer according to the invention in 100 ml of solution.

If the polymers are in the form of aqueous dispersions, corresponding amounts of the dispersion are topped up with ethanol to 100 ml depending on the polymer content of the dispersion, so that the concentration is 1 g of polymer in 100 ml.

The K value is measured in a micro-Ubbelohde capillary type M Ic from Schott.

### Determination of the Droplet Size Distribution (DSD) by Means of Malvern® Scattered Light Analysis

The droplet size distribution was determined using particle size measurement system for detecting liquid aerosols “Malvern® Master Sizer X” (Malvern Instruments Inc., Southborough Mass., USA).

### Measurement Principle:

The measurement principle is the laser light diffraction at the particle, which is suitable not only for spray analysis (aerosols, pump sprays), but also for determining the size of solids, suspensions and emulsions in the size range from 0.1 μm to 2000 μm.

A particle collective (=droplet) is illuminated by a laser. At each droplet, some of the incident laser light is scattered. This light is captured on a multielement detector and the corresponding light energy distribution is determined. This data is used to calculate the corresponding particle distribution using the evaluation software.

### Procedure:

The aerosols were sprayed at a distance of 29.5 cm from the laser beam. The spray cone was at right angles to the laser beam.

Before each measurement, the aerosol cans were attached to a firmly installed holding device, thus meaning that all of the aerosols to be tested were measured at exactly the same distance.

Before the actual particle measurement, a “background measurement” was carried out. By doing so, the effects of dust and other contaminants within the measurement range were eliminated.

The aerosol was then sprayed into the test space. The entire particle volume was detected for a test period of 2 s and evaluated.

### Evaluation:

The evaluation comprises a tabular depiction over 32 class widths from 0.5 μm to 2000 μm and additionally a graphical depiction of the particle size distribution.

Since the spray experiments are an approximately uniform distribution, the mean diameter D (v, 0.5) is given. This numerical value indicates that 50% of the total particle volume measured is below this value.

For readily sprayable aerosol systems in the cosmetics sector, this value is in the range from 30 μm to 80 μm, depending on the polymer content, geometry of the valve and actuator, solvent ratio and amounts of propellant gas.

### Determination of the Setting (Flexural Rigidity):

The setting of polymeric film formers was measured not only by a subjective assessment (hand test), but also physically by measuring the flexural rigidity of thin hair swatches (each about 3 g and 24 cm in length). For this, the weighed, dry hair swatches were dipped into the 3.0% strength by weight polymer solution (solvent: ethanol/water 55:45 w/w), uniform wetting of the hair swatches and distribution of the polymer solution being ensured by dipping and removing the swatches three times and then squeezing them between filter paper. The excess film former solution was then squeezed out between thumb and forefinger and the hair swatches were the swatches shaped by hand so that they had a round cross section. They were dried overnight in a climatically controlled room at 20° C. and 65% relative humidity. The tests were carried out in the climatically controlled room at 20° C. and 65% relative humidity using a stress/strain testing device. The hair swatch was placed symmetrically at the ends on two cylindrical rolls of the sample holder. In exactly the middle, the swatch was then bent from above using a rounded punch ca. 40 mm (breakage of the polymer film). The force required for this (Fmax) was determined using a weighing cell (50 N). Here, one measurement value represents the arithmetic mean from the individual measurements on 5 to 10 identically treated hair swatches. The values ascertained in this way were placed in relation to those for a standard commercial comparison polymer (Amphomer®LV-71) and given in %.

### Determination of the Ability to be Washed Out:

A hair swatch treated with polymer analogously to the determination of the setting was washed in a ca. 37° C.-hot Texapon®NSO solution (6 ml of Texapon®NSO (28% strength) in 11 of warm water) for ca. 15 seconds by dipping it in and squeezing it five times. The hair swatch was then rinsed until clear and treated again in the same way. The hair swatch was then squeezed thoroughly on filter paper and left to dry overnight. The dry hair swatch was put in rollers and analyzed for residues.

### Determination of the Curl Retention

Basic formulation: (aerosol hairspray)

| | |
|---|---|
| 5% by weight | of active ingredient of polymer to be tested |
| | (100% neutr. with AMP) |
| 15% by weight | of ethanol |
| 40% by weight | of water |
| 40% by weight | of dimethyl ether. |

To determine the curl retention, hair swatches ca. 2 g in weight and 15.5 cm in length and comprising mid-brown, Caucasian human hair were used.

### Treatment of the Hair Swatches:

The hair swatches were washed twice with an aqueous Texapon®NSO solution. The hair swatches were then rinsed with warm water until no more foaming was evident and after-rinsed with demineralized water, combed and laid to dry on filter paper.

To prepare a waterwave, the hair swatches are placed for 15 minutes to swell in a solution of ethanol and water (1:1).

The hair swatch was carefully combed before the curl preparation. The hair swatch was attached to a plexiglass rod using a rubber band. It was then combed and wound in the shape of a spiral. Using a cotton cloth and rubber band, the curl was firmly fixed and dried overnight at 70° C. The cooled curl retention swatches were carefully opened and slipped off the plexiglass rod without deforming the waterwave. From a distance of 15 cm, 1.8 g of the aerosol hairspray prepared as mentioned above were sprayed uniformly onto the curl. The curl was rotated evenly during this. In the horizontal position, the curls were dried for 1 h at room temperature. After drying, the curls were secured in a support. Using a ruler, the length of the curls at the start Lₒ was read off and the length extension during humid storage was monitored. After storage for 5 h at 25° C. and 90% relative humidity in the climatically controlled chamber, the length which the curl had reached Lₜ was read off again and the curl retention was calculated according to the following equation:

$$Curl Retention in \% = \frac{L - {L}_{t}}{L - {L}_{0}} * 100$$

Length of the hair (15.5 cm)
L₀=Length of the hair curl after drying
Lₜ=Length of the hair curl after climatic treatment

The mean value from the 5 individual measurements was given as curl retention.

### Determination of the Stickiness

Firstly, a clear, 20% strength by weight ethanolic or ethanolic/aqueous solution of the polymer to be characterized was prepared. In order to obtain a clear solution it was sometimes necessary to neutralize the polymer. A doctor knife (120 μm slit width) was then used to apply a film of the polymer from the ethanolic or ethanolic/aqueous solution on a rectangular glass plate which had a length of ca. 20 cm and a width of ca. 6.5 cm. The polymer film applied thereto had in each case a length of ca. 16 to 18 cm and a width of ca. 5.5 cm.

The film was then dried in the air for ca. 10 hours and then stored in a climatically controlled cabinet for a further 12 hours at 20° C. and 80% relative humidity.

Then, under these conditions, in the climatically controlled cabinet, a plastic carbon ribbon (e.g. Pelikan®2060, 50 mm wide) located on a round rubber punch (diameter 400 mm, Shore A hardness 60±5) was pressed onto the polymer film with a force of about 250 N for 10 seconds.

The amount of black pigment which remains adhering to the polymer film after the punch has been removed corresponds to the stickiness of the film. A visual assessment of the black coloration of the film was made. The assessment scale ranges from 0 to 5, where 0 is not sticky and 5 is very considerably sticky.

### Determination of the Appearance of the Aerosol Formulation

The preparations comprising 5% by weight of the particular polymer neutralized with AMP, 40% by weight of dimethyl ether, 15% by weight of ethanol and 40% by weight of water were poured into a transparent glass aerosol container. The clarity of the resulting liquid/propellant gas mixture was then assessed visually.

### EXAMPLES

The following examples illustrate the invention without limiting it. Unless indicated otherwise, the percentages are percentages by weight.

### ABBREVIATIONS USED

t-BA tert-butyl acrylate
t-BMA tert-butyl(meth)acrylate
HEMA 2-hydroxyethyl methacrylate
HPMA 2-hydroxypropyl methacrylate
MAA methacrylic acid
AA acrylic acid

### Example 1

### tert-butyl acrylate/methacrylic acid 75/25 w/w (comparative example)

In a 2 l polymerization vessel with stirrer and heating and cooling devices, at a temperature from 20 to 25° C.

| | |
|---|---|
| 250 g | of deionized water |
| 0.6 g | of a 15% strength by weight aqueous solution of |
| | sodium lauryl sulfate in deionized water |
| 35 g | of feed II (see below) |

were initially introduced and heated to 45° C. with stirring and under a nitrogen atmosphere. After the temperature had been reached, feed I (see below) was added over the course of 5 minutes.

The mixture was then heated to 80° C. and, while stirring and maintaining the reaction temperature, the remainder of feed II was metered in over the course of 2.5 hours with constant feed streams.

When the feeds were complete, the reaction mixture was stirred for a further hour at 80° C. and then cooled to 60° C.

While maintaining the temperature of 60°, feed III (see below) was added. The mixture was then cooled to 35° C. and, while maintaining the reaction temperature, feed IV (see below) was added over the course of 60 minutes.

### Feed I:

5 g of a 7% strength by weight aqueous solution of sodium persulfate in deionized water

Feed II is an aqueous monomer emulsion prepared from:

| | | % by wt. based |
|---|---|---|
| Initial | | on total amount |
| weight [g] | | of monomer |
| 120 | deionized water | |
| 5 | of a 15% strength by weight aqueous | |
| | solution of sodium lauryl sulfate | |
| | in deionized water | |
| 10 | nonionic emulsifier* | |
| 182 | tert-butyl acrylate | 75 |
| 61 | methacrylic acid | 25 |
| 1.8 | n-dodecyl mercaptan | |

*TweenTM 80, for example, can be used as nonionic emulsifier.

### Preparation of Feed II

The total amount of the 15% strength by weight aqueous solution of sodium lauryl sulfate and then the total amount of the nonionic emulsifier are added with stirring to the initial charge of deionized water. The corresponding amounts of tert-butyl acrylate, methacrylic acid and n-dodecyl mercaptan are added in the order given to the homogeneous solution, which continues to be stirred.

### Feed III:

2 g of a 30% strength by weight solution of hydrogen peroxide in deionized water

### Feed IV:

40 g of a 10% strength by weight solution of ammonium hydrogencarbonate in deionized water

The polymers of the following examples 2 to 7 were synthesized analogously to Example 1, with feed II being chosen accordingly for each example as stated below. Emulsifiers/water/batch size/emulsion preparation/regulator analogous to Example 1.

### Example 8

### Preparation of Polymer 8 (Solution Polymerization in Ethanol)

The following feeds were prepared at 20° C. with stirring:

Feed 1:

| | |
|---|---|
| 123 g | t-BA |
| 55 g | t-BMA |
| 41 g | HEMA |
| 15 g | MAA |
| 5 g | AA |
| 200 g | Ethanol |

Feed 2:

| | |
|---|---|
| 7 g | Wako V 59 |
| 50 g | Ethanol |

At 20° C., a mixture of 300 g of ethanol, 15% of the total amount of feed 1, and 15% of the total amount of feed 2 were prepared. The mixture was heated to 78° C. under atmospheric pressure. After 78° C. had been reached, feed 1 and feed 2 were started at the same time. Feed 1 was metered in over the course of 3 h, and feed 2 was metered in over the course of 4 h with a constant feed stream. The reaction mixture was maintained at 78° C. throughout the entire feed. When feed 2 was complete, the reaction mixture was kept at 78° C. for a further 2 h, then cooled to room temperature.

### Example 9

### Preparation of Polymer 9 (Solution Polymerization in Ethanol/Water with Water-Soluble Starters)

The following feeds were prepared at 20° C. with stirring:

Feed 1:

| | |
|---|---|
| 90 g | MMA |
| 30 g | HEMA |
| 30 g | MAA |
| 200 g | Ethanol |

Feed 2:

| | |
|---|---|
| 3 g | Sodium peroxodisulfate |
| 135 g | Ethanol |
| 102 g | Water |

At 20° C., a mixture of 60 g of ethanol, 45 g of water, 15% of the total amount of feed 1, and 15% of the total amount of feed 2 were prepared. The mixture was heated to 78° C. under atmospheric pressure. After 78° C. had been reached, feed 1 and feed 2 were started at the same time. Feed 1 was metered in over the course of 3 h and feed 2 was metered in over the course of 4 h with a constant feed stream. The reaction mixture was maintained at 78° C. throughout the entire feed. When feed 2 was complete, the reaction mixture was maintained at 78° C. for a further 2 h, then cooled to room temperature.

Details of the monomer composition in percent by weight,

| Polymer from | | | | | | |
|---|---|---|---|---|---|---|
| example | t-BA | t-BMA | HEMA | HPMA | MAA | others |
| 1 (comparative | 75 | | | | 25 | |
| example) | | | | | | |
| 2 | 65 | | 15 | | 20 | |
| 3 | 65 | | 10 | | 25 | |
| 4 | 50 | | 30 | | 20 | |
| 5 | 65 | | | 15 | 20 | |
| 6 | 50 | | | 30 | 20 | |
| 7 | 45 | 20 | 15 | | 20 | |
| 8 | 45 | 20 | 15 | | 15 | 5 AA |
| 9 | 60 | | 20 | | 20 | |

### Application Properties

| Polymer | Clarity | | DSD- | |
|---|---|---|---|---|
| from | in the | Flexural rigidity ** | Malvern | Curl retention |
| example | aerosol*** | [%] | [μm] | [%] |
| 1* | clear | 73 | 73 | 47 |
| 2 | clear | 95 | 70 | 70 |
| 3 | clear | 95 | 69 | 86 |

*Comparative example
** Determination of the setting by means of flexural rigidity method (see Measurement methods)
***VOC55 Aerosol:
5% of the respective polymer, completely neutralized with AMP,
40% DME,
15% ethanol,
40% water;

### Spray Device:

Spray head: Kosmos.020D Wirbel.018″ 21-6443-20 (Precision Valve),

### Valve: DPV 33876 (Precision Valve)

Better setting effect and curl retention are achieved with the polymers of Examples 2 and 3 than with the polymer of Example 1 (comparative example).

### II. Application Examples (AE)

Unless stated otherwise, all of the polymers containing acid groups used are 100% neutralized with AMP. “Water ad 100” means that the amount of water necessary to reach a total amount of 100% is added to the corresponding formulation.

### AE 1:

VOC 55 aerosol hairspray

| | [%] |
|---|---|
| Polymer from Example No. 2 (solid) | 5.00 |
| Dimethyl ether | 40.00 |
| Ethanol | 15.00 |
| Water | ad 100 |

Further additives: Silicone, perfume, antifoam, UV absorber . . .

The example can be repeated in each case with the polymers 3-9 according to the invention and mixtures of these polymers. In each case, a VOC 55 aerosol hairspray with good properties is obtained.

### AE 2:

VOC 55 aerosol hairspray

| | [%] |
|---|---|
| Polymer from Example No. 2 (solid) | 2.00 |
| Polymer from Example No. 3 (solid) | 2.00 |
| Dimethyl ether | 40.00 |
| Ethanol | 15.00 |
| Water | ad 100 |

Further additives: Silicone, perfume, antifoam, UV absorber . . .

The example can be repeated in each case with the polymers 4-9 according to the invention and corresponding mixtures of the individual polymers. In each case, a VOC 55 aerosol hairspray with good properties is obtained.

### AE 3:

VOC 55 aerosol hairspray

| | [%] |
|---|---|
| Polymer from Example No. 2 (solid) | 5.00 |
| Dimethyl ether | 35.00 |
| Propane/butane | 5.00 |
| Ethanol | 15.00 |
| Water | ad 100 |

Further additives: Silicone, perfume, antifoam, UV absorber . . .

The example can be repeated in each case with the polymers 3-9 according to the invention and mixtures of these polymers. In each case, a VOC 55 aerosol hairspray with good properties is obtained.

### AE 4:

Aerosol hairspray with fluorocarbon propellant

| | [%] |
|---|---|
| Polymer from Example No. 2 (solid) | 5.00 |
| Ethanol abs. | ad 100 |
| HFC 152A | 40.00 |

Further additives: Silicone, perfume, antifoam, UV absorber . . .

The example can be repeated in each case with the polymers 3-9 according to the invention and mixtures of these polymers. In each case, an aerosol hairspray with good properties is obtained.

### AE 5:

Aerosol hairspray with fluorocarbon propellants

| | [%] |
|---|---|
| Polymer from Example No. 2 (solid) | 5.00 |
| Dist. water | ad 100 |
| HFC 152A | 10.00 |
| Dimethyl ether | 30.00 |
| Ethanol abs. | 30.00 |

Further additives: Silicone, perfume, antifoam, UV absorber . . .

The example can be repeated in each case with the polymers 3-9 according to the invention and mixtures of these polymers. In each case, an aerosol hairspray with good properties is obtained.

### AE 6:

VOC 55 aerosol hairspray

| | [%] |
|---|---|
| Polymer from Example No. 2 (solid) | 3.00 |
| Ultrahold ® Strong (solid BASF) | 1.00 |
| Dimethyl ether | 40.00 |
| Ethanol | 15.00 |
| + AMP | to pH 8.3 |
| Water | ad 100 |

Further additives: Silicone, perfume, antifoam . . .

The example can be repeated in each case with the polymers 3-9 according to the invention and mixtures of these polymers. In each case, a VOC 55 aerosol hairspray with good properties is obtained.

### AE 7:

VOC 80 aerosol hairspray

| | [%] |
|---|---|
| Polymer from Example No. 2 (solid) | 12.00 |
| Dimethyl ether | 40.00 |
| Ethanol | 40.00 |
| Water | ad 100 |

Further additives: Silicone, perfume, antifoam . . .

The example can be repeated in each case with the polymers 3-9 according to the invention and mixtures of these polymers. In each case, a VOC 80 aerosol hairspray with good properties is obtained.

### AE 8:

Aqueous hand pump spray

| | [%] |
|---|---|
| Polymer from Example No. 2 (solid) | 4.00 |
| Luviset Clear*⁾ (solid) | 1.00 |
| Water | ad 100 |

Further additives: Water-soluble silicone, perfume, antifoam . . .
*⁾Luviset ® Clear: Poly(vinylpyrrolidone/methacrylamide/vinylimidazole), BASF

The example can be repeated in each case with the polymers 3-9 according to the invention and mixtures of these polymers. In each case, an aqueous hand pump spray with good properties is obtained.

### AE 9:

Aqueous/ethanolic setting solution

| | [%] |
|---|---|
| Polymer from Example No. 2 (solid) | 7.0 |
| Dist. water | ad 100 |
| Ethanol | 52.00 |

Further additives: Silicone, perfume, antifoam . . .

The example can be repeated in each case with the polymers 3-9 according to the invention and mixtures of these polymers. In each case, a setting solution with good properties is obtained.

### AE 10:

Ethanolic setting solution

| | [%] |
|---|---|
| Polymer from Example No. 2 (solid) | 7.0 |
| Ethanol | ad 100 |

Further additives: Silicone, perfume, antifoam . . .

The example can be repeated in each case with the polymers 3-9 according to the invention and mixtures of these polymers. In each case, an ethanolic setting solution with good properties is obtained.

### AE 11:

Hair gel

[%]

Phase 1

| | |
|---|---|
| Polymer from Example No. 2 (solid) | 6.0 |
| Water, dist. | ad 50 |

Further additives: Preservative, soluble ethoxylated silicone, perfume . . .

Phase 2:

| | |
|---|---|
| Natrosol HR 250 (5% strength solution) | 50.00 |
| Hydroxyethylcellulose (Hercules) | |

### Preparation:

Phases 1 and 2 are weighed in separately and homogenized. Phase 2 is then slowly stirred into Phase 1. An essentially clear, stable gel is formed.

The example can be repeated in each case with the polymers 3-9 according to the invention and mixtures of these polymers. In each case, a hair-setting gel with good properties is obtained.

### AE 12:

Foam conditioner

| | [%] |
|---|---|
| Polymer from Example No. 2 (solid) | 0.50 |
| Cremophor ® A 25 (ceteareth 25/BASF) | 0.20 |
| Comperlan KD (coamide DEA/Henkel) | 0.10 |
| Propane/butane | 10.00 |
| Further additives: perfume, preservative . . . | |
| Water | ad 100 |

Preparation: Weigh in and Dissolve with Stirring. Bottle and Add Propellant Gas.

The example can be repeated in each case with the polymers 3-9 according to the invention and mixtures of these polymers. In each case, a foam conditioner with good properties is obtained.

### AE 13:

Conditioner shampoo:

| | | [%] |
|---|---|---|
| A) | Texapon NSO 28% strength (sodium laureth sulfate/Henkel) | 50.00 |
| | Comperlan KS (coamide DEA/Henkel) | 1.00 |
| | Polymer from Example No. 2 (solid) | 3.00 |
| | q.s. Perfume oil | |
| B) | Water | 44.5 |
| | Sodium chloride | 1.5 |
| | q.s. Preservative . . . | |

### Preparation:

Phases 1 and 2 are weighed in separately and homogenized. Phase 2 is then slowly stirred into Phase 1. An essentially clear, stable gel is formed.

The example can be repeated in each case with the polymers 3-9 according to the invention and mixtures of these polymers. In each case, a conditioner shampoo with good properties is obtained.

### AE 14:

Standard O/W cream:

| | |
|---|---|
| [%] | CTFA name |

Oil phase:

| | | |
|---|---|---|
| Crempophor A6 | 3.5 | Ceteareth-6 (and) stearyl alcohol |
| Crempophor A25 | 3.5 | Ceteareth-25 |
| Glycerol monostearate | 2.5 | Glycerol stearate |
| s.e. | | |
| Paraffin oil | 7.5 | Paraffin oil |
| Cetyl alcohol | 2.5 | Cetyl alcohol |
| Luvitol EHO | 3.2 | Cetearyl octanoate |
| Vitamin E acetate | 1.0 | Tocopheryl acetate |
| Nip-Nip | 0.1 | Methyl and propyl 4-hydroxybenzoate |
| | | (7:3) |

Water phase:

| | | |
|---|---|---|
| Polymer from Example | 0.6 | |
| No. 2 (Solid) | | |
| Water | 77.0 | |
| 1.2-propylene glycol | 1.5 | Propylene glycol |
| Germall II | 0.1 | Imidazolidinylurea |

### Preparation:

The oil and water phases are weighed in separately and homogenized at a temperature of about 80° C. The water phase is then slowly stirred into the oil phase and cooled slowly to room temperature with stirring.

The example can be repeated in each case with the polymers 3-9 according to the invention and mixtures of these polymers. In each case, a standard O/W cream with good properties is obtained.

### AE 15:

Liquid make-up
A

| | |
|---|---|
| 1.70 | Glycerol stearate |
| 1.70 | Cetyl alcohol |
| 1.70 | Ceteareth-6 |
| 1.70 | Ceteareth-25 |
| 5.20 | Capryl/capric triglyceride |
| 5.20 | Mineral oil |

B

| | |
|---|---|
| q.s. | Preservative |
| 4.30 | Propylene glycol |
| 2.50 | Polymer 2 (solid) |
| ad 100 | dist. water |

C

| | |
|---|---|
| q.s. | Perfume oil |

D

| | |
|---|---|
| 2.00 | Iron oxide |
| 12.00 | Titanium dioxide |

### Preparation:

Heat Phase A and Phase B separately from one another to 80° C. Then mix Phase B into Phase A using a stirrer. Allow everything to cool to 40° C. and add Phase C and Phase D. Homogenize again.

The example can be repeated in each case with the polymers 3-9 according to the invention and mixtures of these polymers. In each case, a make-up with good properties is obtained.

1-9. (canceled)

10. A copolymer obtained by emulsion polymerization, which comprises, in copolymerized form,
a) between 25 and 80% by weight of tert-butyl(meth)acrylate,
b) 2 to 60% by weight of hydroxyalkyl(meth)acrylate,
c) 10 to 40% by weight of an anionic or anionogenic, free-radically polymerizable compound which is or comprises methacrylic acid,
d) 0 to 30% by weight of at least one further free-radically polymerizable compound,
wherein components a) to d) add up to 100% by weight, and
wherein the emulsion polymerization takes place at an inert gas pressure which is higher than a vapor pressure of the emulsion polymerization mixture.

11. The copolymer according to claim 1, which comprises, in copolymerized form,
a) from more than 30 to at most 75% by weight of component a),
b) 5 to 40% by weight of component b),
c) 12 to 35% by weight of component c),
d) 0 to 30% by weight of at least one further free-radically polymerizable compound,
wherein components a) to d) add up to 100% by weight.

12. The copolymer according to claim 1, which comprises in copolymerized form,
a) 40 to 70% by weight of component a),
b) 10 to 35% by weight of component b),
c) 15 to 30% by weight of component c),
d) 0 to 30% by weight of at least one further free-radically polymerizable compound,
wherein components a) to d) add up to 100% by weight.

13. The copolymer according to claim 1, wherein,
a) is tert-butyl acrylate,
b) is hydroxyethyl methacrylate,
c) is methacrylic acid.

14. A method of preparing a copolymer according to claim 1 for cosmetic preparations, comprising polymerizing the emulsion polymerization mixture at an inert gas pressure which is higher than a vapor pressure of the emulsion polymerization mixture.

15. A cosmetic preparation comprising a copolymer according to claim 1.

16. The cosmetic preparation according to claim 6, wherein a fraction of volatile organic components is at most 55% by weight, based on the cosmetic preparation.

17. The cosmetic preparation according to claim 6, wherein the cosmetic preparation also has at least one cosmetically acceptable carrier B, which is chosen from
i) water-miscible organic solvents,
ii) oils, fats, waxes,
iii) esters of C₆-C₃₀-monocarboxylic acids with mono, di- or trihydric alcohols which are different from ii),
iv) saturated acyclic and cyclic hydrocarbons,
v) fatty acids,
vi) fatty alcohols,
vii) propellants (propellant gases) and
viii) mixtures thereof.

18. The cosmetic preparation according to claim 6 in a form of a spray product, wherein the cosmetic preparation is present either in combination with a mechanical pump spray device or in combination with at least one propellant chosen from the group consisting of propane, butane, dimethyl ether, fluorinated hydrocarbons and mixtures thereof.

19. The cosmetic preparation according to claim 8, wherein the water-miscible organic solvent is a C₂-C₄-alkanol.

20. The cosmetic preparation according to claim 8, wherein the water-miscible organic solvent is ethanol.

21. A cosmetic preparation comprising a copolymer, the copolymer comprising, in copolymerized form,
a) from more than 30 to at most 75% by weight of tert-butyl acrylate and tert-butyl methacrylate,
b) 5 to 40% by weight of hydroxyalkyl(meth)acrylate,
c) 12 to 35% by weight of an anionic or anionogenic, free-radically polymerizable compound which is or comprises methacrylic acid, and
d) 0 to 30% by weight of at least one further free-radically polymerizable compound,
wherein components a) to d) add up to 100% by weight, and
wherein the emulsion polymerization takes place at an inert gas pressure which is higher than a vapor pressure of the emulsion polymerization mixture, and
wherein a weight ratio of tert-butyl methacrylate to tert-butyl acrylate is at least 1:2.
